(19) United States
(12) Patent Application Publication
(10) Pub. No.: US 2011/0184303 A1
(43) Pub. Date: Jul. 28, 2011

(54) Methods and Systems Related to Respiration

(75) Inventors: James E. Skinner, Bangor, PA (US); Andriy Batchinsky, San Antonio, TX (US)

(73) Assignee: Nonlinear Medicine, Inc., Boca Raton, FL (US)

(21) Appl. No.: 12/852,329

(22) Filed: Aug. 6, 2010

Related U.S. Application Data
(60) Provisional application No. 61/232,309, filed on Aug. 7, 2009.
(60) Provisional application No. 61/232,365, filed on Aug. 7, 2009.
(60) Provisional application No. 61/232,349, filed on Aug. 7, 2009.
(60) Provisional application No. 61/232,359, filed on Aug. 7, 2009.

(51) Int. Cl. A61B 5/08

(52) U.S. Cl. 600/529

(57) ABSTRACT

Disclosed herein are methods and systems related to respiration. The methods and systems are related to the analysis of a subject's respiration. In some forms the methods and system can use a nonlinear analysis.

### CROSS REFERENCE TO RELATED APPLICATIONS

This application claims the benefit of U.S. Provisional Application Ser. No. 61/232,309 filed on Aug. 7, 2009, U.S. Provisional Application Ser. No. 61/232,365 filed on Aug. 7, 2009, U.S. Provisional Application Ser. No. 61/232,349 filed on Aug. 7, 2009, U.S. Provisional Application Ser. No. 61/232,359 filed on Aug. 7, 2009, all of which are incorporated herein by reference in their entireties.

### STATEMENT REGARDING FEDERALLY FUNDED RESEARCH

Research leading to this invention was funded in part by the United States Government. The U.S. Government has certain rights in this invention.

### BACKGROUND

The present methods and systems are directed to evaluating biological or physical data. More particularly, the present systems and methods are directed to evaluating biological or physical data for detecting and/or predicting abilities, health and clinical outcomes, related to breathing rate.

A medical ventilator delivers gas to a patient's respiratory tract and is often required when the patient is unable to maintain adequate ventilation. Mechanical ventilation is one of the most important therapeutic modalities in the care of critically ill patients. However, the risk for complications increases the longer a patient stays on a ventilator. Accordingly, it is desirable for a patient to be weaned off of a ventilator as soon as possible. Patients that are not physically ready to be removed from the ventilator can get undesirable complications from the weaning process. A method for determining if a patient is ready to be weaned is therefore needed.

### SUMMARY

The objects, advantages and features of the methods disclosed herein will become more apparent when reference is made to the following description taken in conjunction with the accompanying drawings.

Disclosed herein are methods to predict and determine clinical outcomes, by using nonlinear analysis of breathing rates. The results are produced by a nonlinear analysis processing routine using a nonlinear algorithm to analyze the data, e.g. the PD2i algorithm, which is used to detect or predict clinical outcomes.

Another aspect of the methods described herein is to determine a subject's ability to be removed from a ventilator.

### BRIEF DESCRIPTION OF THE DRAWINGS

FIG. 1 shows respiration cycles and determination of respiratory intervals.

FIG. 2 shows PD2i of respiration cycles.

FIG. 3 shows the mean variation of file 102 of respiratory intervals (RR Intervals, upper left) reduced to 180 integers and % N=56.78. Note that rather continuous variation occurred for all 796 intervals. The interval data was supplied by ISR (RR-like data).

FIG. 4 shows the mean variation of file 803 reduced to 180 integers and % N=42.42. The larger respiratory excursions were qualitatively different from those in FIG. 1 in that they were short-long sequences among continuous more normal respiratory interval cycles (small amplitude). The data length was also smaller (n=197 instead of n=796 for file 102).

FIG. 5 shows the respiratory patterns of the peaks of files 102 and 803. Note time base differences (10 sec total for 102, 27 sec total for 803), the steady (102) versus the couplet of short-long respirations in 803, and amplitude differences in the respiratory amplitude.

FIG. 6 shows the summary of the PD2i analysis performed of patients that were successfully removed from a ventilator. N (acc PD2i) means the number of accepted PD2i values for each subject. % N means accepted PD2i divided by all possible PD2i; reject if less than 30%. Note that data are reduced in noise by dividing raw respiratory intervals by a number that adjusts their means to 180 integers. Peak means the peak of accepted PD2i histogram. Mean means the mean accepted PD2i of respiratory intervals. Mean SD is the standard deviation of Mean PD2i. Min PD2i is the minimum PD2i of respiratory intervals. NRi is the number of respiratory intervals. Max NRi is the maximum number of respiratory intervals. Min NRi is the minimum number of respiratory intervals.

FIG. 7 shows the summary of the PD2i analysis performed on patients that could not be removed successfully from a ventilator. N (acc PD2i) means the number of accepted PD2i values for each subject. % N means accepted PD2i divided by all possible PD2i; reject if less than 30%; data are reduced in noise by dividing raw respiratory intervals by a number to adjust the mean to 180 integers; neglect all rejections. Peak means the peak of accepted PD2i histogram. Mean means the mean accepted PD2i of respiratory intervals. Mean SD is the standard deviation of Mean PD2i. Min PD2i is the minimum PD2i of respiratory intervals. NRi is the number of respiratory intervals. Max NRi is the maximum number of respiratory intervals. Min NRi is the minimum number of respiratory intervals.

FIG. 8 shows the data used for the t-test and the p-value calculations. The PASS column indicates patients that were successfully removed from a ventilator. The FAIL column indicates patients that could not be removed from a ventilator. Both columns show mean (bold) and standard deviation of the Min PD2i values. The p-value indicates that the mean PASS and mean FAIL PD2i values are statistically significant.

### DETAILED DESCRIPTION OF THE INVENTION

The inability to tolerate separation from mechanical ventilation or the need for re-intubation occurs in as many as 20% of mechanically ventilated patients and results in increased intensive care unit (ICU) and hospital length of stay, total hospital costs and patient mortality (Rothaar R, et al., Current Topics in Critical Care 2003; 9:56-66; Epstein S, et al., Chest 1997; 112:186-192; Tobin M J., et al., American Review of Respiratory Disease 1986; 134:1111-1118.). Conversely, delaying extubation exposes the patient to the complications and discomfort of unnecessary mechanical ventilation and increased hospital costs (Kollef M, et al., Critical Care Medicine 1997; 25:567-574). Multiple studies have shown that a diverse collection of variables used to predict successful separation from mechanical ventilation perform poorly and add little to the physician's clinical judgment (Meade M, et al., Chest 2001; 120:400 S-424S). Recently, attention has focused on the use of breathing variability as a weaning predictor (El-Khatib M, et al., Intensive Care Medicine 2001; 27:52-58; Engoren M. Critical Care Medicine 1998; 26:1817-1823; Bien M Y, et al., Intensive Care Medicine 2004; 30:241-247; Wysocki M, et al., Critical Care Medicine 2006; 34:2076-2083). Implicit in this approach is that healthy subjects demonstrate a considerable variation in breathing patterns (Tobin M J, et al., Journal of Applied Physiology 1988; 65:309-317; Benchetrit G. Respiration Physiology 2000; 122:123-129; Peng C K, et al., Annals of Biomedical Engineering 2002; 30:683-692); however, in pulmonary disease states, breathing variability is reduced from normal levels (Brack T, et al., American Journal of Respiratory & Critical Care Medicine 2002; 165:1260-1264; Leigh R, et al., Archives of Neurology 1976; 33:356-361; Loveridge B, et al., American Review of Respiratory Disease 1984; 130:730-733). Wysocki and colleagues have postulated that respiratory variability is related to pulmonary load balance and that increased loading reduces breathing variability (Wysocki M, et al., Critical Care Medicine 2006; 34:2076-2083). Data from healthy human volunteers as well as two recent weaning studies support this hypothesis (Bien M Y, et al., Intensive Care Medicine 2004; 30:241-247; Wysocki M, et al., Critical Care Medicine 2006; 34:2076-2083; Tobin M J, et al., Journal of Applied Physiology 1988; 65:309-317; Brack T, et al., American Journal of Respiratory & Critical Care Medicine 1998; 157:1756-1763; Preas H L, et al., American Journal of Respiratory & Critical Care Medicine 2001; 164:620-626; Jubran A, et al., American Journal of Respiratory & Critical Care Medicine 2000; 162:1202-1209; Jubran A, et al., American Journal of Respiratory & Critical Care Medicine 1997; 156:1129-1139; Brack T, et al., American Journal of Respiratory & Critical Care Medicine 1997; 155:1341-1348; Shore E, et al., Journal of Applied Physiology 1984; 59:1605-1615) although contrasted findings have been reported (El-Khatib M, et al., Intensive Care Medicine 2001; 27:52-58; Engoren M. Critical Care Medicine 1998; 26, Gilbert R, et al., Chest 1974; 65:152-157).

Breathing variability may be quantified by methods that involve nonlinear dynamical analysis, i.e. PD2i. A nonlinear system is one whose behavior is not simply a summation of inputs into the system; nonlinearity is a fundamental characteristic of normal physiological data (Godin P J, et al., Critical Care Medicine 1996; 24:1107-1116). These methods are distinct from variance, which measures dispersion about a mean, and take into account the nonlinear physiologic response to stimuli. As such nonlinear methods can provide insight into organ system interconnectivity and regulatory control (Godin P J, et al., Critical Care Medicine 1996; 24:1107-1116; Pincus S M. Mathematical Biosciences 1994; 122:161-181).

Previously a panel of nonlinear analysis tools was applied to the assessment of waveforms and established that lower cardiovascular regulatory complexity as sampled from electrocardiographic signal irregularity was associated with adverse outcomes in pre-hospital trauma patients (Batchinsky A I, et al., J Trauma 2007; 63:512-518). Described herein are methods and utilitilizing nonlinear analysis tools in the assessment of perturbation in the respiratory domain. One such tool is the PD2i algorithm. Another tool is Sample Entropy (SampEn) which is a relatively new family of statistics measuring regularity of nonlinear, clinical, and experimental time series data. It examines the data for similar epochs (groups of consecutive points of the same length) in which more frequent and more similar epochs yield lower values of this metric (Richman J S, et al., American Journal of Physiology—Heart & Circulatory Physiology 2000; 278:H2039-2049). This allows comparison of patterns to determine which is the most regular (i.e. least complex). In addition, the assessment of signal irregularity was complemented with methodologically distinct waveform analysis tools such as those derived from analysis of signal amplitude distribution as a function of time (Zochowski M, et al., Physical Review E 1997; 56:3725-3727); entropy of symbol dynamics distributions (Hao B. Physica D 1991; 51:161-176; Palazzolo J A, et al., Am J Physiol 1998; 274:H1099-1105); and assessment of baseline shifts, or stationarity of the signal (Palazzolo J A, et al., Am J Physiol 1998; 274:H1099-1105).

Described herein are methods that measure the regularity of breathing patterns of intubated patients undergoing spontaneous breathing trials (SBTs) using a comprehensive analysis of respiratory waveforms. Patients who successfully separate from mechanical ventilation are likely to have a more irregular breathing pattern than those who fail extubation as measured by methodologically different nonlinear metrics.

A. Definitions

1. A, an, the

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

2. Cell

The term “cell” as used herein also refers to individual cells, cell lines, or cultures derived from such cells. A “culture” refers to a composition comprising isolated cells of the same or a different type. The term co-culture is used to designate when more than one type of cell are cultured together in the same dish with either full or partial contact with each other.

3. Clinical Outcomes

A clinical outcome is a documented clinical event, in a subject, such as needing to be placed on a ventilator or taken off a ventilator, that is made by a physician. The clinical outcomes can be any outcome, including those disclosed herein.

4. Comprise

Throughout the description and claims of this specification, the word “comprise” and variations of the word, such as “comprising” and “comprises,” means “including but not limited to,” and is not intended to exclude, for example, other additives, components, integers or steps.

5. Computer Readable Media, Computer Program Product, Processors. Computer Usable Memory, Computer Systems

In some embodiments, instructions stored on one or more computer readable media that, when executed by a system processor, cause the system processor to perform the methods described above, and in greater detail below. Further, some embodiments can include systems implementing such methods in hardware and/or software. A typical system can include a system processor comprising one or more processing elements in communication with a system data store (SDS) comprising one or more storage elements. The system processor can be programmed and/or adapted to perform the functionality described herein. The system can include one or more input devices for receiving input from users and/or software applications. The system can include one or more output devices for presenting output to users and/or software applications. In some embodiments, the output devices can include a monitor capable of displaying to a user graphical representation of the described analytic functionality.

The described functionality can be supported using a computer including a suitable system processor including one or more processing elements such as a CELERON, PENTIUM, XEON, CORE 2 DUO or CORE 2 QUAD class microprocessor (Intel Corp., Santa Clara, Calif.) or SEMPRON, PHENOM, OPTERON, ATHLON X2 or ATHLON 64×2 (AMD Corp., Sunnyvale, Calif.), although other general purpose processors could be used. In some embodiments, the functionality, as further described below, can be distributed across multiple processing elements. The term processing element can refer to (1) a process running on a particular piece, or across particular pieces, of hardware, (2) a particular piece of hardware, or either (1) or (2) as the context allows. Some implementations can include one or more limited special purpose processors such as a digital signal processor (DSP), application specific integrated circuits (ASIC) or a field programmable gate arrays (FPGA). Further, some implementations can use combinations of general purpose and special purpose processors.

The environment further includes a system data store (SDS) that could include a variety of primary and secondary storage elements. In one preferred implementation, the SDS would include registers and RAM as part of the primary storage. The primary storage can in some implementations include other forms of memory such as cache memory, non-volatile memory (e.g., FLASH, ROM, EPROM, etc.), etc. The SDS can also include secondary storage including single, multiple and/or varied servers and storage elements. For example, the SDS can use internal storage devices connected to the system processor. In implementations where a single processing element supports all of the functionality a local hard disk drive can serve as the secondary storage of the SDS, and a disk operating system executing on such a single processing element can act as a data server receiving and servicing data requests.

It will be understood by those skilled in the art that the different information used in the systems and methods for respiratory analysis as disclosed herein can be logically or physically segregated within a single device serving as secondary storage for the SDS; multiple related data stores accessible through a unified management system, which together serve as the SDS; or multiple independent data stores individually accessible through disparate management systems, which can in some implementations be collectively viewed as the SDS. The various storage elements that comprise the physical architecture of the SDS can be centrally located or distributed across a variety of diverse locations.

6. Computer Network

A computer network or like terms are one or more computers in operable communication with each other.

7. Computer Implemented

Computer implemented or like terms refers to one or more steps being actions being performed by a computer, computer system, or computer network.

8. Computer Program Product

A computer program product or like terms refers to product which can be implemented and used on a computer, such as software.

9. Control

The terms “control” or “control levels” or “control cells” are defined as the standard by which a change is measured, for example, the controls are not subjected to the experiment, but are instead subjected to a defined set of parameters, or the controls are based on pre- or post-treatment levels. They can either be run in parallel with or before or after a test run, or they can be a pre-determined standard.

10. Expiration Phase (EP)

Expiration phase and like terms refers to the period during a respiration cycle in which air is moving out of the lungs.

11. Digitized Electrocardiogram (ECG)

A digitized electrocardiogram refers to an ECG that has been produced by digitizing the analog data of an ECG.

12. Good Ability

“Good ability” or the like terms refer to a high expectance, based on a subject's ability, of accomplishing a task based on particular indicators i.e. high PD2i value, low PD2i value, strength, speed, age, weight. “Good ability” does not mean that a subject will or can accomplish the task.

13. Higher

The terms “higher,” “increases,” “elevates,” or “elevation” or variants of these terms, refer to increases above basal levels, e.g., as compared to a control. The terms “low,” “lower,” “reduces,” or “reduction” or variation of these terms, refer to decreases below basal levels, e.g., as compared to a control. For example, basal levels are normal in vivo levels prior to, or in the absence of, or addition of an agent such as an agonist or antagonist to activity.

14. High PD2i Value

“High PD2i value” or the like term or phrase refers to a PD2i value that is equal or higher than the ventilator removal standard. For example, a high PD2i value can be equal or higher than 3.50, 3.30, 3.25, 3.15, 3.05, 2.95, 2.85, 2.75, 2.65, 2.55, 2.45 or 2.35. In another example, a high PD2i value can be equal or higher than 3.25, 3.15, 3.05, 2.95, 2.85 or 2.75. In another example, a high PD2i value can be equal or higher than 3.15. In another example, a high PD2i value can be equal or higher than 2.75.

15. Identification of a Clinical State

A clinical state is for example, alive, dead, healthy, sick, dying, stable etc. The identification of a clinical state, refers to determining at a moment in time, what clinical state a subject is in. In certain embodiments, one can determine what clinical state a subject will likely be in.

16. Inhibit

By “inhibit” or other forms of inhibit means to hinder or restrain a particular characteristic. It is understood that this is typically in relation to some standard or expected value, in other words it is relative, but that it is not always necessary for the standard or relative value to be referred to. For example, “inhibits phosphorylation” means hindering or restraining the amount of phosphorylation that takes place relative to a standard or a control.

17. Inspiration Phase (IP)

Inspiration phase and like terms refers to the period during a repiration cycle in which air is moving into the lungs.

18. Lower the Level of Noise

The noise refers to the amplitude of random noise within data. It can be large spikes superimposed on the real data (large outliers) or small low-level random noise superimposed on each data point. Lowering the noise refers to reducing the amplitude of the random noise added at each data point.

19. Low PD2i Value

“Low PD2i value” or the like term or phrase refers to a PD2i value that is lower than the ventilator removal standard. For example, a low PD2i value can be lower than 3.15, 3.00, 2.85, 2.75, 2.55, 2.35 or 2.15. In another example a low PD2i value can be lower than, 2.75, 2.55, 2.35 or 2.15. In another example a low PD2i value can be lower than 2.75. In another example, a low PD2i value can be lower than 2.35.

20. Nonlinear Analysis

A nonlinear analysis is based on a nonlinear mathematical model and it is usually considered vis-a-vis a linear stochastic (statistical) model. Through modern usage it has come to mean a deterministic model of any exponent that is not a probabilistic model with an exponent of 1 (linear). Nonlinear analysis is very sensitive to noise content. For example, a nonlinear analysis can be based on the PD2i algorithm.

21. Obtaining

Obtaining as used in the context of data or values, such as RRi data or values refers to acquiring this data or values. It can be acquired, by for example, collection, such as through a machine, such as an ECG machine or a respiratory machine. It can also be acquired by downloading or getting data that has already been collected, and for example, stored in a way in which it can be retrieved at a later time.

22. Optional

“Optional” or “optionally” means that the subsequently described event or circumstance may or may not occur, and that the description includes instances where said event or circumstance occurs and instances where it does not.

23. Outputting Results

Outputting or like terms means an analytical result after processing data by an algorithm.

24. PD2i Algorithm

PD2i “scales as” ∝ log C(n, r, nref*)/log-R where ∝ means “scales as,” C is the count of vector difference lengths within a step size of R in the correlation integral for PD2i in which n equals the data length, r equals the scaling range, and nref* equals a location of the reference vector for estimating the scaling region slope of log C/log r in a restricted small log-R range that is devoid of the effects of non-stationary data.

25. Poor Ability

“Poor ability” or the like terms refer to a low expectance, based on a subject's ability, of accomplishing a task based on particular indicators i.e. high PD2i value, low PD2i value, strength, speed, age, weight. “Poor ability” does not mean that a subject will not or can not accomplish the task.

26. Prexpiration Phase (PEP)

A prexpiration phase or like terms refers to the period during a respiration cycle prior to an expiration phase in which there is no inspiration or expiration.

27. Preinspiration Phase (PIP)

A preinspiration phase or like terms refers to the period during a respiration cycle prior to an inspiration phase in which there is no inspiration or expiration.

28. Prevent

By “prevent” or other forms of prevent means to stop a particular characteristic or condition. Prevent does not require comparison to a control as it is typically more absolute than, for example, reduce or inhibit. As used herein, something could be reduced but not inhibited or prevented, but something that is reduced could also be inhibited or prevented. It is understood that where reduce, inhibit or prevent are used, unless specifically indicated otherwise, the use of the other two words is also expressly disclosed. Thus, if inhibits phosphorylation is disclosed, then reduces and prevents phosphorylation are also disclosed.

29. Real-Time R-R Interval (RRi) Values

A real-time R-R interval value refers to the real-time between consecutive R-wave peaks, typically provided in milliseconds. A real-time R-R interval is given in a time unit. A real-time R-R interval is obtained by first counting the number of data points between R-wave peaks observed in the digitized data from an ECG and then multiplying each point count by a conversion factor that converts the point count to a real time value. For example, if the digitization rate occurs at 500 Hz, i.e. 500 data points produced per second, and the heart rate is 60 bpm, then there will be one heart beat per second, and so then there will be approximately 500 data points between R-wave peaks, which when turned to a real-time R-R interval would require multiplying the 500 data points by conversion factor of 2 msec/data-point to yield 1000 milliseconds. This conversion factor is actually the sampling period (i.e., the amount of time in each data point at that frequency of digitization).

30. R-R Interval (RRi) Data

Any data that reflects the amount of time between two events as they happen in real time. RRi data could be obtained between two breaths or two heart beats, for example.

31. Ranges

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data are provided in a number of different formats, and that this data, represents endpoints and starting points, and ranges for any combination of the data points. For example, if a particular datum point “10” and a particular datum point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

32. Reduce

By “reduce” or other forms of reduce means lowering of an event or characteristic. It is understood that this is typically in relation to some standard or expected value, in other words it is relative, but that it is not always necessary for the standard or relative value to be referred to. For example, “reduces phosphorylation” means lowering the amount of phosphorylation that takes place relative to a standard or a control.

33. Respiration

Respiration or like terms refers to the act of a subject breathing.

34. Respiration Cycle

A Respiration cycle or like terms refers to the actions taking place during one breath of a subject. The respiration cycle, as discussed herein includes an inspiration phase, a preexpiration phase, an expiration phase, and a preinspiration phase. Typically, a respiration cycle will have one inspiration phase, one preexpiration phase, one expiration phase, and one preinspiration phase in a single cycle (a IP-PEP-EP-PIP). However, because respiration can be consciously controlled it is understood that this typical four phase system can be altered such that for example, a subject has an inspiration phase, does not intake air in for a period of time, and then inspires still more air prior to the preexpiration phase, the expiration phase, and the preinspiration phase. Thus, this type of cycle would have had a IP-PIP-IP-PEP-EP-PIP cycle.

35. Respiratory Rate, Breathing Rate

Respiratory rate or breathing rate and like terms represents the number of breaths a subject takes during a certain period of time. Often this can be given in breaths per minute.

36. Respiratory Record

A respiratory record or like terms is any collection of respiratory data.

37. Respiratory Mark (RM)

A respiratory mark and like terms refers to a point during a respiratory cycle. For example, respiratory mark could be 1 second after the start of the inspiration phase, or at the start of inspiration phase, or one collected data point after the start of inspiration phase. A respiratory mark is used to identify the same points on successive respiratory cycle, and two consecutive respiratory marks at the same point in the cycle produce a respiratory mark interval.

38. Respiratory Mark Interval (RMi)

A respiratory mark interval or like terms refers to the time or number of data points between two consecutive respiratory marks.

39. Respiratory Mark Interval Data Series

A respiratory mark interval data series or like terms refers to a collection of respiratory mark intervals.

40. Respiratory Data Series

A respiratory data series or like terms refers to any collection of respiratory data.

41. Respirogram, Respiratory Trace

A respirogram or respiratory trace refers to any graphical presentation of respiration data.

42. Sampling Period

The sampling period refers to the sample and hold time of each time interval of the digitizer. Also see Real-time R-R Interval above.

43. Subject

“Subject” like terms refer to an individual. Thus, the “subject” can include, for example, domesticated animals, such as cats, dogs, etc., livestock (e.g., cattle, horses, pigs, sheep, goats, etc.), laboratory animals (e.g., mouse, rabbit, rat, guinea pig, etc.) and mammals, non-human mammals, primates, non-human primates, rodents, birds, reptiles, amphibians, fish, and any other animal. In one aspect, the subject is a mammal such as a primate or a human. The subject can be a non-human.

44. Successive Respiratory Mark

A successive respiratory mark or like terms refers to same mark in the next respiratory cycle.

45. Tidal Volume

The Tidal volume or like terms is the lung volume representing the normal volume of air displaced between normal inhalation and exhalation when extra effort is not applied.

46. Treating

“Treating” or “treatment” does not mean a complete cure. It means that the symptoms of the underlying disease are reduced, and/or that one or more of the underlying cellular, physiological, or biochemical causes or mechanisms causing the symptoms are reduced. It is understood that reduced, as used in this context, means relative to the state of the disease, including the molecular state of the disease, not just the physiological state of the disease. In certain embodiments, a treatment can actually do unforeseen harm to a subject.

47. Therapeutically Effective

The term “therapeutically effective” means that the amount of the composition used is of sufficient quantity to ameliorate one or more causes or symptoms of a disease or disorder. Such amelioration only requires a reduction or alteration, not necessarily elimination. The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

48. Ventilator Removal Standard

“Ventilator removal standard” or the like terms refers to a PD2i value. The PD2i value can be an empirically determined PD2i value. The PD2i value can be a Mean PD2i value or a Min PD2i value. The PD2i value can be determined by analyzing PD2i values from subjects that were successfully removed from a ventilator, from subjects that were not successfully removed from a ventilator or from a combination thereof. The analysis of the PD2i values can be done by averaging the Mean PD2i values or Min PD2i values. For example, the ventilator removal standard can be less than 5, 4.5, 4.0, 3.5, 3.0, 2.5, 2.0, 1.5, 1.0, or 0.5. In another example the ventilator removal standard can be between 5.00-4.50, 4.50-4.00, 4.00-3.50, 3.50-3.30, 3.30-3.15, 3.15-3.00, 3.00-2.85, 2.85-2.75, 2.75-2.65, 2.65-2.55, 2.55-2.45, 2.45-2.35, 2.35-2.25, 2.25-2.15, 2.15-2.00, 2.00-1.85, 1.85-1.75 or 1.75-1.65. In some forms the ventilator removal standard can be 3.50-3.30, 3.30-3.15, 3.15-3.00, 3.00-2.85, 2.85-2.75, 2.75-2.65, 2.65-2.55, 2.55-2.45 or 2.45-2.35. In some forms the ventilator removal standard can be less than 2.0 or 1.8.

49. Ventilation Rate

Ventilation rate and like terms represents the rate at which gas enters and leaves the lung.

50. Vital Capacity

Vital capacity or like terms is the maximum volume of air that a person can exhale after maximum inhalation.

B. Methods and Apparatus

The respiratory rate (RR) and respiration cycle of an individual can be measured using a variety of mechanisms, including electronically, physically, digitally, and manually. As discussed herein, the respiratory cycle is made up of inspiration phase, an expiration phase, and a preexpiration phase and a preinspiration phase.

The simplest way to measure the RR is to manually note the upward movement of the chest.

There are devices which will measure movement of the chest through pressure sensitivity, through for example, a chest strap. A full fabric chest garment, known as a Bioharness™, produced by Zephyr technology ltd can also be used. Pyroelectic polymer films, (PEP) have also been used to measure non-intubated respiratory rates. Another example is a spirometer. A spirometer is an apparatus which measures the amount, volume, of inspiration or expiration. It typically uses a precise pressure transducer to measure respiration flow rates. A spirometer produces an output called a kymograph trace. The trace can be used to calculate vital capacity, vital volume, breathing rate, and ventilation rate.

A medical (mechanical) ventilator delivers gas to a patient's respiratory tract and is often required when the patient is unable to maintain adequate ventilation. Mechanical ventilation is one of the most important therapeutic modalities in the care of critically ill patients. Known ventilators typically include a pneumatic system that delivers and extracts gas pressure, flow and volume characteristics to the patient and a control system (typically consisting of knobs, dials and switches) that provides the interface to the treating clinician. Optimal support of the patient's breathing requires adjustment by the clinician of the pressure, flow, and volume of the delivered gas as the condition of the patient changes. Such adjustments, although highly desirable, are difficult to implement with known ventilators because the control system demands continuous attention and interaction from the clinician.

Further, patients requiring ventilatory assistance must overcome airway resistance in the breathing circuit during exhalation. This resistance, combined with the stiffness of the lungs and the thoracic cage under certain pathological conditions, imposes a significant workload upon a patient whose reserves may already be compromised by underlying disease processes.

Mechanical ventilation is used, among other reasons, for patients with acute respiratory distress, temporarily after surgery, or while sedated or pharmacologically paralyzed. Most patients can be removed from mechanical ventilation and resume breathing on their own. Some patients require long-term mechanical ventilation (i.e., quadriplegia) and, in some cases, mechanical ventilation is considered life-support for patients who would otherwise die.

1. Ventilation and Breathing Assist Systems

Mechanical ventilation replaces or supports the normal ventilatory lung function of the patient. Although mechanical ventilation is usually used for acute illness or injury in an intensive care setting, patients who require long-term mechanical ventilation can receive it at home under the supervision of a physician and home health agency. The patient must have a tracheostomy for long-term therapy.

There are several modes of mechanical ventilation, each offering different advantages and disadvantages. Many can be used in conjunction with one another. In a ventilator assist situation, where the ventilator is assisting the breathing of the subject, the initiation of the assist can occur through breath termination, breath initiation, or breath volume. Microprocessor technology has enabled the combination of various ways of initiation because the ventilator is able to handle data analysis combinations of all of these modes as well as flow-sensing, which controls the ventilator breath based on the flow-rate of gas versus a specific volume, pressure, or time.

Examples of ventilators can be found in U.S. Pat. Nos. 6,152,135, 6,082,357, 5,474,062, 5,315,989, 5,307,795, 6,584,973, 6,390,091, 7,497,215, for example, and are herein incorporated in their entireties by reference at least for machines, systems, and apparati for ventilation, breathing assist devices.

a) Control Ventilation (CV)

CV delivers the preset volume or pressure regardless of the patient's own inspiratory efforts. This mode is used for patients who are unable to initiate a breath. If it is used with spontaneously breathing patients, they must be sedated and/or pharmacologically paralyzed so they do not breathe out of synchrony with the ventilator.

b) Assist-Control Ventilation (A/C) or Continuous Mandatory Ventilation (CMV)

Both A/C and CMV deliver the preset volume or pressure in response to the patient's inspiratory effort, but will initiate the breath if the patient does not do so within the set amount of time. This mode is used for patients who can initiate a breath but who have weakened respiratory muscles. The patient may need to be sedated to limit the number of spontaneous breaths as hyperventilation can occur in patients with high respiratory rates.

c) Synchronous Intermittent Mandatory Ventilation (SIMV)

SIMV delivers the preset volume or pressure and preset respiratory rate while allowing the patient to breathe spontaneously. The vent initiates each breath in synchrony with the patient's breaths. SIMV is used as a primary mode of ventilation as well as a weaning mode. (During weaning, the preset rate is gradually reduced, allowing patients to slowly regain breathing on their own.) The disadvantage of this mode is that it may increase the work of breathing and respiratory muscle fatigue. Breathing spontaneously through ventilator tubing has been compared to breathing through a straw.

d) Positive-End Expiratory Pressure (PEEP)

PEEP is positive pressure that is applied by the ventilator at the end of expiration. This mode does not deliver breaths but is used as an adjunct to CV, A/C, and SIMV to improve oxygenation by opening collapsed alveoli at the end of expiration. Complications from the increased pressure can include decreased cardiac output, lung rupture, and increased intracranial pressure.

e) Pressure Support Ventilation (PSV)

PSV is preset pressure that augments the patient's spontaneous inspiration effort and decreases the work of breathing. The patient completely controls the respiratory rate and tidal volume. PSV is used for patients with a stable respiratory status and is often used with SIMV during weaning.

f) Intermittent Positive Pressure Breathing (IPPB)

IPPB is a form of assisted ventilation in which compressed oxygen is delivered under positive pressure into the patient's airway until a preset pressure is reached. Exhalation is passive. The cycle is repeated for the ordered number of breaths. IPPB is often used for a short time after a patient is removed from of a ventilator to promote maximal lung expansion and to help clear secretions.

g) Neurally Adjusted Ventilatory Assist (NAVA)

Neurally Adjusted Ventilatory Assist (NAVA) identifies a mode of mechanical ventilation where the ventilator is controlled directly by the subject's own neural impulses controlling breathing. The respiration neural control signal originates in the respiratory center, and is transmitted through the phrenic nerve to excite the diaphragm. These signals can be monitored by means of electrodes mounted on a nasogastric feeding tube and positioned in the esophagus at the level of the diaphragm. As respiration increases and the respiratory center requires the diaphragm for more effort, the degree of ventilatory support needed is identified. This means that the subject's respiratory center is in direct control of the mechanical support needed on a breath-by-breath basis, and any variation in the neural respiratory demand is responded to by the appropriate corresponding change in ventilatory assistance.

h) Breath Termination

In a volume-cycled ventilator the ventilator delivers a pre-set volume of gas with each breath. Once the specified volume of breath is delivered, the positive pressure is terminated after a certain specified time period. Both pressure and volume modes of ventilation have their respective limitations. Many manufacturers provide a mode or modes that utilize some functions of each. These modes are flow-variable, volume-targeted, pressure-regulated, time-limited modes (for example, pressure-regulated volume control—PRVC). This means that instead of providing an exact tidal volume each breath, a target volume is set and the ventilator will vary the inspiratory flow at each breath to achieve the target volume at the lowest possible peak pressure. The inspiratory time limits the length of the inspiratory cycle and therefore the I:E ratio. Pressure regulated modes such as PRVC or Auto-flow (Draeger) can most easily be thought of as turning a volume mode into a pressure mode with the added benefit of maintaining more control over tidal volume than with strictly pressure-control.

i) Breath Initiation

The other method of classifying mechanical ventilation is based on how to determine when to start giving a breath. Similar to the termination classification noted above, microprocessor control has resulted in a myriad of hybrid modes that combine features of the traditional classifications. Note that most of the timing initiation classifications below can be combined with any of the termination classifications listed above.

2. Problems with Removal from Ventilator

There can be several problems for a patient when removed from a ventilator. One complication of mechanical ventilation can be the patients' dependence on the ventilator and the inability to wean them off. For example, weaning inspiratory muscle disuse can develop in a patient because a minimum level of inspiratory muscle activity must be present with the proportional assist ventilation (PAV) modality. If PAV is used throughout the illness, there would be no period in which the central control mechanisms are inactive (apnea). Central respiratory dysfunction is common in the weaning period and can be due, in part, to protracted inactivity of the respiratory centers produced by machine settings that promote apnea with other modalities of ventilatory support. The lesser likelihood of central and peripheral muscle dysfunction facilitates weaning.

The longer a patient is dependent on a ventilator the bigger the risk is of complications. Therefore, the sooner a subject is removed from the ventilator the better, but premature discontinuation of mechanical ventilation can compromise gas exchange and lead to problems with reintubation (Maclntyre, N. R., Cook, D. J., et al. (2001). Evidence-based guidelines for weaning and discontinuing ventilatory support are available. (A collective task force facilitated by the American College of Chest Physicians, the American Association for Respiratory Care and the American College of Critical Care Medicine. Chest, 120(6 Suppl), 375S). In fact, nearly one-third of ICU patients on mechanical ventilation cannot be weaned on the first attempt (Burns, S. (2005). AACN procedure manual for critical care (5th ed.). Philadelphia: Elsevier Saunders). Other complications with premature discontinuation include stress. Subjects assisted by mechanical ventilation are often weak and worried. Stress could potentially worsen their mental or physical condition. Since premature discontinuation of mechanical ventilation can cause complications; a method for determining a subject's ability to be removed from a ventilator would be useful.

Disclosed herein are methods to determine a subject's ability to be removed from a ventilator.

3. Nonlinear Algorithms and RRi and RMi Values Determination Related to Physiological Data, Such as PD2i

An RR interval is the time or the space between two successive events, such as the time between the peaks of a heart ECG trace or the time between two breaths. Successive RR intervals can be used to produce an R-R series i.e. from heart rate or breathing rate intervals. A Analog signal must be turned into a digital signal and it must be done at particular rate, Hz. For example 187 data points or 500 data points or 1000 datapoints per second, which corresponds to a 187-, 500-, and 1000-Hz respectively. To get to a time interval in a digital environment, the cycle rate is multiplied by a factor to bring it to a 1000 milliseconds. Once this conversion is made, the Hz rate is multiplied by the conversion factor, this is the realtime RRi data.

To get the time associated with a particular datapoint, the datapoint number is multiplied by a conversion factor, which is defined as 1000 divided by the Hz rate. Thus, for example, in a series recorded at 500 Hz, the 450^{th} datapoint was recorded at 900 ms after the onset of the recording. Accordingly, one can convert an entire data series from “(datapoint number, datapoint value)” format to “(time of datapoint, datapoint value)” format by multiplying each datapoint number by the conversion factor, e.g., for 500 Hz data: (1, 17 mV), (2, 12 mV), (3, 16 mV) . . . etc., becomes (2 ms, 17 mV), (4 ms, 12 mV), (6 ms, 16 mV), . . . etc.

In certain methods and systems the nonlinear algorithm used to analyze nonlinear data, including variation, including in certain systems and methods, variation in the RR interval can be the PD2i algorithm, which is disclosed in for example, U.S. Pat. No. 7,276,026 for “Method and system for detecting and/or predicting cerebral disorders” to Skinner, U.S. Pat. No. 7,076,288 for “Method and system for detecting and/or predicting biological anomalies to Skinner, U.S. Pat. No. 5,720,294 for “PD2I electrophysiological analyzer” to Skinner, and U.S. Pat. No. 5,709,214 for “PD2i electrophysiological analyzer” to Skinner, as well as PCT Publication No. WO 2008/028004 for “Automated Noise Reduction System for Predicting Arrythmic Deaths by Skinner and PCT Publication No. WO 2006/076543 for “Knowledge Determination System” to Skinner, all of which are incorporated by reference herein in their entireties at least for material related to PD2i and its use in biological systems.

The model for the PD2i is C(r,n,ref*,) R expD2, where ref* is an acceptable reference point from which to make the various n-dimensional reference vectors, because these will have a scaling region of maximum length PL that meets the linearity (LC) and convergence (CC) criteria. Because each ref* begins with a new coordinate in each of the m-dimensional reference vectors and because this new coordinate could be of any value, the PD2i′s can be independent of each other for statistical purposes.

The PD2i algorithm limits the range of the small log-R values over which linear scaling and convergence are judged by the use of a parameter called Plot Length. The value of this entry determines for each log-log plot, beginning at the small log-R end, the percentage of points over which the linear scaling region is sought.

In non-stationary data, the small log-R values between a fixed reference vector (i-vector) in a subepoch that is, say, a sine wave, when subtracted from multiple j-vectors in, say, a Lorenz subepoch, will not make many small vector-difference lengths, especially at the higher embedding dimensions. That is, there will not be abundant small log-R vector-difference lengths relative to those that would be made if the j-vector for the Lorenz subepoch was instead in a sine wave subepoch. When all of the vector-difference lengths from the non-stationary data are mixed together and rank ordered, only those small log-R values between subepochs that are stationary with respect to the one containing the reference vector will contribute to the scaling region, that is, to the region that will be examined for linearity and convergence. If there is significant contamination of this small log-R region by other non-stationary subepochs, then the linearity or convergence criterion will fail, and that estimate will be rejected from the accepted PD2i mean.

The PD2i algorithm introduced to the art the idea that the smallest initial part of the linear scaling region should be considered if data non-stationarities exist (i.e. as they always do in biological data). This is because when the j-vectors lie in a subepoch of data that is the same species as that the i-vector (reference vector) is in, then and only then will the smallest log-R vectors be made abundantly, that is, in the limit or as data length becomes large. Thus, to avoid contamination in the correlation integral by species of data that are non-stationary with respect to the species the reference vector is in, one should look only at the slopes in the correlation integral that lie just a short distance beyond the initial small log-R “floppy tail”.

The “floppy tail” is the very smallest log-R range in which linear scaling does not occur due to the lack of points in this part of the correlation integral resulting from finite data length. Thus, by restricting the PD2i scaling to the smallest part of the log-R range above the “floppy tail,” the PD2i algorithm becomes insensitive to data non-stationarities. Note that the D2i always uses the whole linear scaling region, which always will be contaminated if non-stationarities exist in the data.

4. Methods and Machines for Analyzing Breathing

Provided are methods, systems, machines, and computer readable media for analyzing respiratory rates. These methods can include steps for the determination of actions to be taken with a subject, such as removal of a subject from a ventilator or placing a subject on a ventilator.

In one embodiment, the systems and methods employ the PD2i algorithm. In some forms the systems and methods employing the PD2i algorithm work on the R to R interval of BRI. For example, typically a respiratory rate recording is digitized and an algorithm is run or a person visually observes to determine the respiratory mark (RM) and the successive number of data points between each pair of respiratory marks, which forms the respiratory mark interval. A respiratory mark can be any point between the initiation of a breath, on inspiration to the end of expiration of a respiratory cycle. A preferred method for placing respiratory marks is performed by a person using high magnification of respirograms to avoid noise being added by errors in automated algorithms. For example, in certain embodiments, a person can detect the abrupt upward trajectory produced by an inspiration as well as an algorithm that must deal with noise in the baseline.

A respiratory mark interval gives the count of the number of data points that lie between successive respiratory marks. Then the algorithm can multiply those data counts by a constant which converts them to realtime in milliseconds. If digitized, for example, at 187 HZ this means there will be 187 datapoints each second in time. So the count of data points between the respiratory marks, can be 185, 192, datapoints, etc. Then to convert to realtime in milliseconds one must multiply the data point counts 1000 divided 187, which equals 5.34 for an RM series with a mean of 60 breaths/Min. Then, the PD2i is performed on the “milliseconds.” It looks at the variation between the breathing marks. So if all RM intervals were equally separated in time, sat at 1000 ms, then there would be no variation and one would have a PD2i equal to zero. As disclosed herein, the lower the PD2i, the more likely there is a problem with the breathing, for example, coming off of a ventilator would be problematic. Having variation is good. Of note, if there is increased noise in the data, however, that leads to the variation, this is a spurious increase in the PD2i (since the PD2i of noise is infinite) and which could give a false PD2i reading.

The noise correction algorithm disclosed in U.S. Pat. No. 7,276,026 for “Method and system for detecting and/or predicting cerebral disorders” to Skinner, and U.S. Pat. No. 7,076,288 for “Method and system for detecting and/or predicting biological anomalies” reduces this noise by reducing the amplitude of the R wave to half. This increases the specificity of the PD2i calculation.

The PD2i can also be run on the count of data points between the R waves in an ECG rather than RR intervals of millisecond units. As disclosed in herein that multiplying number also increases the noise.

This concept could be used any time one uses the PD2i. Minimum noise in the data file is preferred, and in some cases essential.

Disclosed herein, when one is doing nonlinear analysis, one should do it on the data points between breaths and not after they have been multiplied by the real-time factor, as that only increases the noise in the data stream, that is already increased due to the descretization error going up as digitization rate goes down.

According to exemplary embodiments, a methods and systems have been developed to reduce or eliminate noise in real-time R-R intervals (RRi) values to nonlinear analytical measures, using PD2i, wherein the data is only available in real-time RRi values. In preferred embodiments, a method for calculating the PD2i of BRI has been developed when data is only available in real-time RRi values. Methods and systems for this can be found in U.S. Application No. 61/153,245 for METHODS AND SYSTEMS FOR REAL-TIME RRi VALUES PD2i OF HEARTBEAT INTERVALS filed on Feb. 17, 2009, which is herein incorporated by reference at least for material related to RRi intervals.

The R-R interval refers to the actual number of milliseconds that occurred between the successive heartbeats when the original digital recording of the data was made. For example, if the heart beat was once per second (60b/m) and the data were digitized at 1000 Hz, 500 Hz or 187 Hz, the R-R intervals would be the count of data points between R-waves times a factor that depends on the digitization rate:

R−R=1000 data points×1000 msec/1000 dp=1000 msec (for 1000 Hz digitization),

or

R−R=500 data points×1000 msec/500 dp=1000 msec (for 500 Hz digitization),

or

R−R=187 data points×1000 msec/187 dp=1000 msec (for 187 Hz digitization).

The noise due to descretization error goes up as the digitization rate goes down. Descretization error is the error caused by the sample period: the longer the sampling period (i.e. the longer the time between successive datapoints), the more uncertainty there is in two issues. (1) it is uncertain whether the specific event targeted (e.g. the peak of the R-wave) occurred between any 2 datapoints at all (“it was missed”) and (2) even though it is certain that the event occurred between 2 given datapoints, it is uncertain to when it occurred—it could be off (in milliseconds) by as much as the sampling period divided by 2, since, in the worst case scenario, it could have occurred exactly in the middle of the sampling period and it was declared to have occurred at either one of the surrounding datapoints. For example:

Descretization error=2/1000=0.002 (i.e., 0.2%) for 1000 Hz data

Descretization error=2/500=0.004 (i.e., 0.4%) for 500 Hz data

Descretization error=2/187=0.0100 (i.e., 1%) for 187 Hz data

PD2i analysis of the R-R intervals has traditionally been done by performing the analysis using the real-time RRi values. As stated herein, the real-time RRi values are generated by multiplying the data with a factor that is dependent on the digitization rate. For example, if the RRi values were taken at 187 Hz the factor or sampling period that is multiplied by the data is determined by 1000 Hz/187 Hz=5.345. This means that the noise is enhanced in the data series and the PD2i analysis can become inaccurate. Many R-R interval detectors work in this manner by counting the number of data-points between R-wave peaks and then multiplying them by a factor that is dependent on the digitization rate to bring the values to the real-time.

Conventional nonlinear analysis, for example using the PD2i algorithm, of real-time RRi values takes place after the number of data point values has been multiplied by the sampling period, e.g. 1× for 1000 Hz, 2× for 500 Hz, and 5.345× for 187 Hz. The direct result of the multiplication is an increase in the level of noise (by the multiplication factor just described) in the data file. However, nonlinear analysis, such as PD2i, assumes a low level noise as it performs the analysis. This type of nonlinear analysis therefore increases the noise in the data stream which is already increased due to the descretization error going up as the digitization rate goes down. A high level of noise from the analysis can potentially lead to misrepresentation or apriori rejection of the data which can lead to an inaccurate analysis. The current method described herein eliminates the increases of noise so that nonlinear analysis, using PD2i algorithm, can more accurately analyze real-time RRi values.

The method described herein divides the real-time values with the sampling period before the nonlinear, PD2i, analysis is performed so that the analysis is performed only on the lowest level of noise in the data. That is, by dividing the real-time values (data point values multiplied by the sampling period) with the sampling period, the data point values becomes yet again the lowest level noise in the data.

Also described herein is a method of analysis of real-time R-R intervals (RRi) values to nonlinear analytical measures, using PD2i. As described previously conventional nonlinear analysis, for example using the PD2i algorithm, of real-time RRi values takes place after the number of data point values has been multiplied by the sampling period, e.g. 1× for 1000 Hz, 2× for 500 Hz, and 5.345× for 187 Hz. Described herein is a method where the data undergoes nonlinear analysis, using PD2i, before they have been multiplied by the sampling period. The nonlinear analysis would then take place between the RRi data-point count values rather than after the multiplication by the sampling period.

a) BRI Data as RRi Values for Nonlinear Analysis

The breathing rate intervals (BRI) can also function as RR intervals. The BRI can be obtained electrically, digitally, or manually. Both breathing rate and heartbeat rate are controlled by centers in the brainstem that are often called the autonomic nervous system because the regulation is without conscious awareness. The other parts of the brain project into these autonomic nuclei and can provide more conscious control of the rhythms of breathing and heartbeating (e.g., holding of breath, increased heartbeating when frightened). Each rhythm when recorded electronically goes up and down in repetitious cycles that enable the intervals between cycles to be measured. These intervals have a variation that is measured by the PD2i. If they were random, then PD2i would be infinite; if they were homogeneously the same, then PD2i would be 0. Physiological data has interval variations that produce PD2i values ranging between these two extremes. The PD2i values at rest and without conscious control tend to range around the number of independent regulators of the variations in the intervals—that is what the PD2i measures, the number of degrees of freedom in the interval variation.

Disclosed herein are machines, apparati, and systems, which are designed to perform the various methods disclosed herein. It is understood that these can be multipurpose machines having modules and/or components dedicated to the performance of the disclosed methods. For example, a medical ventilator can be modified as described herein so that it contains a module and/or component which for example, a) produces a respiratory record, which identifies one or more respiratory marks, identifies one or more respiratory mark intervals, creates a respiratory mark interval data series, and/or performs a nonlinear analysis, such as a PD2i analysis alone or in any combination. In particular, the modules and components within the ventilator responsible for determining when to begin assisting a breath, can be linked to the modules and/or components responsible for identifying and/or manipulating a respiratory mark. In certain embodiments the respiratory mark, can be the moment or determined by the moment the ventilator starts or stops a breath cycle.

Thus, the methods and systems herein can have the data, in any form uploaded by a person operating a device capable of performing the methods disclosed herein. The methods can also be associated with the breathing assist devices as described herein, either incorporated into these systems or being on device which is connected to them.

5. Systems, Machines, and Computer Readable Medium

In addition, or instead, the functionality and approaches discussed above, or portions thereof, can be embodied in instructions executable by a computer, where such instructions are stored in and/or on one or more computer readable storage media. Such media can include primary storage and/or secondary storage integrated with and/or within the computer such as RAM and/or a magnetic disk, and/or separable from the computer such as on a solid state device or removable magnetic or optical disk. The media can use any technology as would be known to those skilled in the art, including, without limitation, ROM, RAM, magnetic, optical, paper, and/or solid state media technology.

6. Applications and Methods

As discussed herein, the health of a subject can be determined using PD2i analysis. A PD2i algorithm calculates the complexity and degrees of freedom in the data set which can be used to determine the health of a subject. Physiological data (such as ECG or breathing rate interval) with low PD2i values indicate poor health and physiological data (such as ECG or breathing rate interval) with high PD2i values indicate good health. Low PD2i values indicate that there is little complexity in the data series, meaning, for example, that there is little variation in the breathing rate interval or in the heart rate interval. Typically, a healthy subject has much complexity and variation in breathing rate interval and/or in the heart rate interval resulting in high PD2i values.

A subject's ability to be removed from a ventilator can be determined using PD2i analysis. For example, a PD2i algorithm can analyze BRI data, ECG data or both. Low PD2i values indicate poor ability to be removed from a ventilator and high PD2i values indicate good ability to be removed from a ventilator. The PD2i values can be used to determine if a subject could attempt to be removed from a ventilator.

The recovery after surgery can be monitored using PD2i. For example, the PD2i analysis can be calculated based on BRI data, ECG data or both. Low PD2i values indicate a poor recovery from surgery while high PD2i values indicate a good recovery of surgery.

Disclosed herein are methods of analyzing a subject's respiration comprising, performing a nonlinear analysis of a respiratory mark interval data series.

Also disclosed herein are methods of analyzing a subject's respiration comprising; receiving a respiratory record, wherein the record contains at least two successive respiration marks; measuring the time interval between at least two successive respiration marks producing a respiratory mark interval; performing step b) for n successive marks producing a respiratory mark interval data series; performing a nonlinear analysis on the respiratory mark interval data series; and outputting results from the nonlinear analysis.

Also disclosed herein are methods of analyzing respiration of a subject comprising, recommending the performance of any of the methods disclosed herein to be performed, alone in any combination with any other characteristic herein.

Also disclosed herein are methods comprising the steps of receiving an output from any of the methods disclosed herein and recommending the removal of the subject from a ventilator alone in any combination with any other characteristic herein.

Also disclosed herein are one or more computer readable media storing program code that, upon execution by one or more computer systems, cause the computer systems to perform the any of the methods disclosed herein, alone in any combination with any other characteristic herein.

Also disclosed herein are computer program products comprising a computer usable memory adapted be executed to implement the any of the methods disclosed herein.

Also disclosed herein are computer program products, comprising a computer usable medium having a computer readable program code embodied therein, said computer readable program code adapted to be executed to implement a method for generating the nonlinear analysis of the respiratory mark interval data series of any of the methods disclosed herein, said method comprising further comprising: providing a system, wherein the system comprises distinct software modules, and wherein the distinct software modules comprise a logic processing module, a configuration file processing module, a data organization module, and a data display organization module.

Also disclosed herein are respiratory analysis systems, the system comprising: a data store capable of storing respiratory data; a system processor comprising one or more processing elements, the one or more processing elements programmed or adapted to: receive respiratory data comprising at least two successive respiration marks; store the respiratory data in the data store; measure the time interval between at least two successive respiration marks producing a respiratory mark interval; repeat step 3) for n successive marks producing a respiratory mark interval data series; identify a Mean PD2i for the data series; compare the Mean PD2i for the data series to a ventilator removal standard; and output a ventilator recommendation based upon the comparison of the Mean PD2i with the ventilator removal standard.

Also disclosed herein are systems capable of performing any of the methods disclosed herein.

Also disclosed herein are computer-readable mediums having stored thereon instructions that, when executed on a programmed processor perform any of the methods disclosed herein.

Also disclosed herein are methods wherein a subject's ability to be removed from a ventilator is determined based on the mean minimum PD2i value, wherein a high mean minimum PD2i value indicates good ability and a low mean minimum PD2i value indicates poor ability of a subject to be removed from a ventilator, alone in any combination with any other characteristic herein.

In some forms, the methods can be computer implemented methods. The computer implemented methods can be any computer implemented method as described elsewhere herein.

In some forms, the methods can further comprise the step of outputting results from the nonlinear analysis.

In some forms, the methods can further comprise producing the respiratory data series before analyzing the series alone in any combination with any other characteristic herein. In some forms, the respiratory data series can be produced from measuring the interval between each successive respiratory mark of a respiratory record.

In some forms, receiving the respiratory record can comprise receiving the respiratory record from a storage medium. In some forms, receiving the respiratory record can comprise receiving the record from a computer system. In some forms receiving the respiratory record can comprise receiving the record from a breathing assistance system. In some forms, receiving the respiratory record can comprise receiving the respiratory record via a computer network.

In some forms, the respiration mark can occur at the start of an inspiration phase. In some forms, the respiration mark can occur within a specified time period of the start of an inspiration phase. For example, the respiration mark can occur within 1 second of the start of an inspiration phase. In another example the respiration mark can occur within 0.5 or 1.5 second of an inspiration phase. In some forms, the respiration mark can occur within the first observable data point of the start of an inspiration phase as identified on a respiratory trace. The first observable data point can be detected using a computer system. In some forms, the respiration mark can occur when a breathing assistance system begins assisting a breath. The breathing assistant system can be any breathing assistant system as described elsewhere herein. For example, the breathing assistant system can be CV, A/C, CMV, SIMV, PEEP, PSV, IPPB or NAVA.

In some forms, the time interval can be obtained by converting a data series. In some forms, the respiratory data series can comprise at least 10 to 10,000 members in the series (e.g., Ni>10^{PD2i}). In some forms, the respiratory data series can comprise at least 1, 2, 3, 4, 5, 10, 25, 50, 100, 250, 500, 1,000, 2,500, 5,000, 7,500, 10,000 or any members in the series (e.g., Ni>10^{PD2i}).

In some forms, the nonlinear analysis involves using the PD2i algorithm. In some forms, the nonlinear analysis involves using the Min PD2i value. In some forms, the nonlinear analysis involves using the PD2i algorithm and its Min PD2i value. In some forms, the nonlinear analysis involves using the Mean PD2i value. In some forms, the nonlinear analysis involves using the PD2i algorithm and its Mean PD2i value. In some forms, the nonlinear analysis is the PD2i algorithm alone in any combination with any other characteristic herein. For example, the PD2i algorithm can be performed on multiple physiological data, such as respiration or the heart rate, as described elsewhere herein.

In some forms, the methods further comprise identifying a Mean PD2i or Min PD2i for the data series. In some forms, the methods further comprise the step of comparing the Mean PD2i or Min PD2i for the data series to a ventilator removal standard. In some forms, the ventilator removal standard can be an empirically determined number. For example, the ventilator removal standard can be determined by analyzing PD2i values from subjects that were successfully removed from a ventilator, from subjects that were not successfully removed from a ventilator or from a combination thereof. In some forms, the analysis of the PD2i values can be done by averaging the Mean PD2i values or Min PD2i values. For example, the ventilator removal standard can be the average Mean PD2i value or average Min PD2i value of subjects that were successfully removed from a ventilator. In another example, the ventilator removal standard can be the average Mean PD2i value or average Min PD2i value from subjects that were not successfully removed from a ventilator. In another example, the ventilator removal standard can be the average of the average Mean PD2i or Min PD2i values for both subjects that successfully were removed from a ventilator and for subjects that were not successfully removed from the ventilator (i.e. (average Min PD2i for subjects that were successfully removed+average Min PD2i for subjects that were not successfully removed)/2). For example, the ventilator removal standard can be less than 5, 4.5, 4.0, 3.5, 3.0, 2.5, 2.0, 1.5, 1.0, or 0.5. In another example the ventilator removal standard can be between 5.00-4.50, 4.50-4.00, 4.00-3.50, 3.50-3.30, 3.30-3.15, 3.15-3.00, 3.00-2.85, 2.85-2.75, 2.75-2.65, 2.65-2.55, 2.55-2.45, 2.45-2.35, 2.35-2.25, 2.25-2.15, 2.15-2.00, 2.00-1.85, 1.85-1.75 or 1.75-1.65. In some forms the ventilator removal standard can be 3.50-3.30, 3.30-3.15, 3.15-3.00, 3.00-2.85, 2.85-2.75, 2.75-2.65, 2.65-2.55, 2.55-2.45 or 2.45-2.35. In some forms the ventilator removal standard can be less than 2.0 or 1.8.

In some forms the methods can further comprise the step of recommending the removal of the subject from the ventilator if the PD2i is greater than the ventilator removal standard, alone in any combination with any other characteristic herein. For example, if the ventilator removal standard is 2.75 and the subjects PD2i is 3.17, then the subject is recommended to be removed from the ventilator, the recommendation can be based on the PD2i value alone or in any combination with any other characteristic described herein.

In some forms, the methods can further comprise performing a PD2i analysis on an RRi data series produced from an ECG from the subject alone in any combination with any other characteristic herein.

In some forms, the computer programs of any of the methods disclosed herein, can comprise a logic processing module, a configuration file processing module, a data organization module, and data display organization module, that are embodied upon a computer readable medium.

In some forms, any of the methods disclosed herein can further comprise a computerized system configured for performing the method.

In some forms, any of the methods disclosed herein, can further comprise the outputting the results from the nonlinear analysis.

In some forms, the system can receive the respiratory data from a breathing assistance system. In some forms, the system can receive the respiratory data via a computer network. In some forms, the system can further comprise a breathing assistance system alone in any combination with any other characteristic herein.

In some forms, high PD2i can be equal or higher than the ventilator removal standard. For example, a high PD2i value can be equal or higher than 3.50, 3.30, 3.25, 3.15, 3.05, 2.95, 2.85, 2.75, 2.65, 2.55, 2.45 or 2.35. In another example, a high PD2i value can be equal or higher than 3.25, 3.15, 3.05, 2.95, 2.85 or 2.75. In another example, a high PD2i value can be equal or higher than 3.15. In another example, a high PD2i value can be equal or higher than 2.75.

In some forms, low PD2i can be lower than the ventilator removal standard. For example, a low PD2i value can be lower than 3.15, 3.00, 2.85, 2.75, 2.55, 2.35 or 2.15. In another example a low PD2i value can be lower than, 2.75, 2.55, 2.35 or 2.15. In another example a low PD2i value can be lower than 2.75. In another example, a low PD2i value can be lower than 2.35.

In some forms, one, two, three or more PD2i values from different physiological sources can be used to determine, predict or monitor a subject as described elsewhere herein. For example, health, the ability to be removed from a ventilator, or clinical outcomes can be predicted or determined by using a PD2i value calculated from the breathing rate interval as described elsewhere herein or PD2i value calculated from ECG data as described elsewhere herein. A subject's health, ability to be removed from a ventilator, or clinical outcomes can also be determined using the PD2i value calculated from the breathing rate interval and the PD2i value calculated from ECG data as described elsewhere herein. The two PD2i values can be compared. Multiple comparable PD2i values can increase the confidence of a prediction, determination or clinical outcome. For example, if both the BRI and ECG PD2i values are high, then it is highly likely that a subject can be removed from a ventilator. A subject's health can also be determined using the PD2i value calculated from the breathing rate interval, the PD2i value calculated from ECG data and the PD2i from another physiological data set.

In some forms, two or more similar PD2i values from different physiological data series can increase the certainty in health, ability or clinical outcome analysis compared to one PD2i value. For example, upon analysis subject has a low PD2i value calculated from the breathing rate interval. A conclusion from the data could be that the subject has poor ability to be removed from a ventilator. A low PD2i value calculated simultaneously from the subject's ECG data would give more confidence in the determination of the poor ability to be removed from a ventilator. Two or more similar PD2i values for different physiological data series can improve the confidence in determining or predict health, ability to be removed from a ventilator, and clinical outcomes of a subject.

However, one PD2i value, based on a physiological data set, could be more important then another PD2i value, based on another physiological data set. For example, when determining if a subject can be removed from a ventilator; the PD2i based on the breathing rate interval could be more important then the PD2i based on ECG data.

The determination if a PD2i value is low or high can be differently based on the nature of the physiological data series. For example, a PD2i value can be considered low when originated from ECG data but the same PD2i value could at the same time not be considered low if originated from another physiological data series. The nature of the complexity of the physiological data series determines if a PD2i value is low or high. For example, in a healthy subject, the natural complexity can be different for different physiological data. The ECG data can for instance be naturally more complex compared to other physiological data. A PD2i value for ECG data can be considered low while the same PD2i value for other physiological data can be considered high. Each individual physiological data set has its individual parameters if a PD2i value is considered low or high.

In some forms, PD2i values can for example be calculated based on a subject's breathing rate intervals as described elsewhere herein.

In some forms, PD2i values can for example be calculated based on ECG data as described elsewhere herein.

In some forms, PD2i values related to breathing can be used to determine a subject's health, such as coming off a ventilator. RRi, such as RMi, real-time values, from the breathing rate interval or ECG data, can be used to calculate a PD2i value using the PD2i algorithm. A subject with high PD2i values on RMi data is more likely to be in better health, such as ability to come off a ventilator, than a subject with low PD2i values on RMi data.

A subject with a PD2i value, such as a Mean PD2i or Min PD2i value, of >5.0, is more likely to be in better health, such as ability to come off a ventilator, than a subject with a PD2i value, such as a Mean PD2i value or Min PD2i value, of 5.0-4.5; a subject with a PD2i value, such as a Mean PD2i value or Min PD2i value of 5.0-4.5 is more likely to be in better health, such as ability to come off a ventilator, than a subject with a PD2i value, such as a Mean PD2i value or Min PD2i value of 4.5-4.0; a subject with a PD2i value, such as a Mean PD2i value or Min PD2i value of 4.5-4.0 is more likely to be in better health, such as ability to come off a ventilator, than a subject with a PD2i value, such as a Mean PD2i value or Min PD2i value Mean PD2i value or Min PD2i value of 4.0-3.5; a subject with a PD2i value, such as a Mean PD2i value or Min PD2i value of 4.0-3.5 is more likely to be in better health, such as ability to come off a ventilator, than a critically with a PD2i value, such as a Mean PD2i value or Min PD2i value of 3.5-3.0; a subject with a PD2i value, such as a Mean PD2i value or Min PD2i value of 3.5-3.0 is more likely to be in better health, such as ability to come off a ventilator, than a subject with a PD2i value, such as a Mean PD2i value or Min PD2i value of 3.0-2.5; a subject with a PD2i value, such as a Mean PD2i value or Min PD2i value of 3.0-2.5 is more likely to be in better health, such as ability to come off a ventilator, than a subject with a PD2i value, such as a Mean PD2i value or Min PD2i value of 2.5-2.0; a subject with a PD2i value, such as a Mean PD2i value or Min PD2i value of 2.5-2.0 is more likely to be in better health, such as ability to come off a ventilator, than a subject with a PD2i value, such as a Mean PD2i value or Min PD2i value of 2.0-1.5; a subject with a PD2i value, such as a Mean PD2i value or Min PD2i value of 2.0-1.5 is more likely to be in better health, such as ability to come off a ventilator, than a subject with a PD2i value, such as a Mean PD2i value or Min PD2i value of 1.5-1.0; a subject with a PD2i value, such as a Mean PD2i value or Min PD2i value of 1.5-1.0 is more likely to be in better health, such as ability to come off a ventilator, than a subject with a PD2i value, such as a Mean PD2i value or Min PD2i value of 1.0-0.5; a subject with a PD2i value, such as a Mean PD2i value or Min PD2i value of 1.0-0.5 is more likely to be in better health, such as ability to come off a ventilator, than a subject with a PD2i value, such as a Mean PD2i value or Min PD2i value of 0.5-0.0.

A subject with a PD2i value, such as a Mean PD2i value or Min PD2i value, of >5.0 is more likely to be in better health, such as ability to come off a ventilator, than a subject with a PD2i value, such as a Mean PD2i value or Min PD2i value, of 5.0-0.0; a subject with a PD2i value, such as a Mean PD2i value or Min PD2i value, of 5.0-4.5 is more likely to be in better health, such as ability to come off a ventilator, than a subject with a PD2i value, such as a Mean PD2i value or Min PD2i value, of 4.5-0.0; a subject with a PD2i value, such as a Mean PD2i value or Min PD2i value, of 4.5-4.0 is more likely to be in better health, such as ability to come off a ventilator, than a subject with a PD2i value, such as a Mean PD2i value or Min PD2i value, of 4.0-0.0; a subject with a PD2i value, such as a Mean PD2i value or Min PD2i value, of 4.0-3.5 is more likely to be in better health, such as ability to come off a ventilator, than a critically with a PD2i value, such as a Mean PD2i value or Min PD2i value, of 3.5-0.0; a subject with a PD2i value, such as a Mean PD2i value or Min PD2i value, of 3.5-3.0 is more likely to be in better health, such as ability to come off a ventilator, than a subject with a PD2i value, such as a Mean PD2i value or Min PD2i value, of 3.0-0.0; a subject with a PD2i value, such as a Mean PD2i value or Min PD2i value, of 3.0-2.5 is more likely to be in better health, such as ability to come off a ventilator, than a subject with a PD2i value, such as a Mean PD2i value or Min PD2i value, of 2.5-0.0; a subject with a PD2i value, such as a Mean PD2i value or Min PD2i value, of 2.5-2.0 is more likely to be in better health, such as ability to come off a ventilator, than a subject with a PD2i value, such as a Mean PD2i value or Min PD2i value, of 2.0-0.0; a subject with a PD2i value, such as a Mean PD2i value or Min PD2i value, of 2.0-1.5 is more likely to be in better health, such as ability to come off a ventilator, than a subject with a PD2i value, such as a Mean PD2i value or Min PD2i value, of 1.5-0.0; a subject with a PD2i value, such as a Mean PD2i value or Min PD2i value, of 1.5-1.0 is more likely to be in better health, such as ability to come off a ventilator, than a subject with a PD2i value, such as a Mean PD2i value or Min PD2i value, of 1.0-0.0; a subject with a PD2i value, such as a Mean PD2i value or Min PD2i value, of 1.0-0.5 is more likely to be in better health, such as ability to come off a ventilator, than a subject with a PD2i value, such as a Mean PD2i value or Min PD2i value, of 0.5-0.0.

As described elsewhere herein, the PD2i of ECG data can be used in combination with the PD2i of RMi data as described above to determine the health of a critically injured subject. For example, the health can be determined for subjects that have been critically injured, underwent surgery, or had trauma.

PD2i values can be used to determine a subject's recovery after surgery. RRi real-time values, from the breathing rate interval or ECG data, can be used to calculate a PD2i value, such as a Mean PD2i value or Min PD2i value, using the PD2i algorithm. A subject with high PD2i value, such as a Mean PD2i value or Min PD2i value, after surgery is more likely to be in better health, such as ability to come off a ventilator, than a subject with low PD2i value, such as a Mean PD2i value or Min PD2i value. A subject with a PD2i value, such as a Mean PD2i value or Min PD2i value, of >5.0 after surgery is more likely to be in better health, such as ability to come off a ventilator, than a subject with a PD2i value, such as a Mean PD2i value or Min PD2i value, of 5.0-4.5; a subject with a PD2i value, such as a Mean PD2i value or Min PD2i value, of 5.0-4.5 after surgery is more likely to be in better health, such as ability to come off a ventilator, than a subject with a PD2i value, such as a Mean PD2i value or Min PD2i value, of 4.5-4.0; a subject with a PD2i value, such as a Mean PD2i value or Min PD2i value, of 4.5-4.0 after surgery is more likely to be in better health, such as ability to come off a ventilator, than a subject with a PD2i value, such as a Mean PD2i value or Min PD2i value, of 4.0-3.5; a subject with a PD2i value, such as a Mean PD2i value or Min PD2i value, of 4.0-3.5 after surgery is more likely to be in better health, such as ability to come off a ventilator, than a subject with a PD2i value, such as a Mean PD2i value or Min PD2i value, of 3.5-3.0; a subject with a PD2i value, such as a Mean PD2i value or Min PD2i value, of 3.5-3.0 after surgery is more likely to be in better health, such as ability to come off a ventilator, than a subject with a PD2i value, such as a PD2i value, such as a Mean PD2i value or Min PD2i value, of 3.0-2.5; a subject with a PD2i value, such as a Mean PD2i value or Min PD2i value, of 3.0-2.5 after surgery is more likely to be in better health, such as ability to come off a ventilator, than a subject with a PD2i value, such as a Mean PD2i value or Min PD2i value, of 2.5-2.0; a subject with a PD2i value, such as a Mean PD2i value or Min PD2i value, of 2.5-2.0 after surgery is more likely to be in better health, such as ability to come off a ventilator, than a subject with a PD2i value, such as a Mean PD2i value or Min PD2i value, of 2.0-1.5; a subject with a PD2i value, such as a Mean PD2i value or Min PD2i value, of 2.0-1.5 after surgery is more likely to be in better health, such as ability to come off a ventilator, than a subject with a PD2i value, such as a Mean PD2i value or Min PD2i value, of 1.5-1.0; a subject with a PD2i value, such as a Mean PD2i value or Min PD2i value, of 1.5-1.0 after surgery is more likely to be in better health, such as ability to come off a ventilator, than a subject with a PD2i value, such as a Mean PD2i value or Min PD2i value, of 1.0-0.5; a subject with a PD2i value, such as a Mean PD2i value or Min PD2i value, of 1.0-0.5 after surgery is more likely to be in better health, such as ability to come off a ventilator, than a subject with a PD2i value, such as a Mean PD2i value or Min PD2i value, of 0.5-0.0. As described elsewhere herein, the PD2i value, such as a Mean PD2i value or Min PD2i value, of ECG data can be used in combination with the PD2i value, such as a Mean PD2i value or Min PD2i value, of breathing rate interval data as described above to determine the health of subject after surgery.

PD2i values can be used to determine a subject's recovery after surgery. RRi real-time values, from the breathing rate interval or ECG data, can be used to calculate a PD2i value, such as a Mean PD2i value or Min PD2i value, using the PD2i algorithm. A subject with high PD2i value, such as a Mean PD2i value or Min PD2i value, after surgery is more likely to be in better health, such as ability to come off a ventilator, than a subject with low PD2i value, such as a Mean PD2i value or Min PD2i value. A subject with a PD2i value, such as a Mean PD2i value or Min PD2i value, of >5.0 after surgery is more likely to be in better health, such as ability to come off a ventilator, than a subject with a PD2i value, such as a Mean PD2i value or Min PD2i value, of 5.0-0.0; a subject with a PD2i value, such as a Mean PD2i value or Min PD2i value, of 5.0-4.5 after surgery is more likely to be in better health, such as ability to come off a ventilator, than a subject with a PD2i value, such as a Mean PD2i value or Min PD2i value, of 4.5-0.0; a subject with a PD2i value, such as a Mean PD2i value or Min PD2i value, of 4.5-4.0 after surgery is more likely to be in better health, such as ability to come off a ventilator, than a subject with a PD2i value, such as a Mean PD2i value or Min PD2i value, of 4.0-0.0; a subject with a PD2i value, such as a Mean PD2i value or Min PD2i value, of 4.0-3.5 after surgery is more likely to be in better health, such as ability to come off a ventilator, than a subject with a PD2i value, such as a Mean PD2i value or Min PD2i value, of 3.5-0.0; a subject with a PD2i value, such as a Mean PD2i value or Min PD2i value, of 3.5-3.0 after surgery is more likely to be in better health, such as ability to come off a ventilator, than a subject with a PD2i value, such as a Mean PD2i value or Min PD2i value, of 3.0-0.0; a subject with a PD2i value, such as a Mean PD2i value or Min PD2i value, of 3.0-2.5 after surgery is more likely to be in better health, such as ability to come off a ventilator, than a subject with a PD2i value, such as a Mean PD2i value or Min PD2i value, of 2.5-0.0; a subject with a PD2i value, such as a Mean PD2i value or Min PD2i value, of 2.5-2.0 after surgery is more likely to be in better health, such as ability to come off a ventilator, than a subject with a PD2i value, such as a Mean PD2i value or Min PD2i value, of 2.0-0.0; a subject with a PD2i value, such as a Mean PD2i value or Min PD2i value, of 2.0-1.5 after surgery is more likely to be in better health, such as ability to come off a ventilator, than a subject with a PD2i value, such as a Mean PD2i value or Min PD2i value of 1.5-0.0; a subject with a PD2i value, such as a Mean PD2i value or Min PD2i value, of 1.5-1.0 after surgery is more likely to be in better health, such as ability to come off a ventilator, than a subject with a PD2i value, such as a Mean PD2i value or Min PD2i value, of 1.0-0.0; a subject with a PD2i value, such as a Mean PD2i value or Min PD2i value, of 1.0-0.5 after surgery is more likely to be in better health, such as ability to come off a ventilator, than a subject with a PD2i value, such as a Mean PD2i value or Min PD2i value, of 0.5-0.0. As described elsewhere herein, the PD2i value, such as a Mean PD2i value or Min PD2i value PD2i of ECG data can be used in combination with the PD2i value, such as a Mean PD2i value or Min PD2i value PD2i of breathing rate interval data as described above to determine the health of subject after surgery.

As described elsewhere herein, the Mean PD2i value or Min PD2i value PD2i of ECG data can be used in combination with the Mean PD2i value or Min PD2i value PD2i of breathing rate interval data as described above to determine if a subject is likely to be removed from a ventilator.

In some forms, the methods described herein can be used on subjects that are supported by mechanical ventilation. In some forms, the mechanical ventilation is a ventilation or assistant breathing system. In some forms, the ventilation or assistant breathing system is assisting the breathing of the subject. For example, the ventilation or assistant breathing system can be CV, A/C, CMV, SIMV, PEEP, PSV, IPPB or NAVA.

U.S. Patent Application No. 61/232,365 entitled “Method of Predicting Medical Events”, filed Aug. 7, 2009 and U.S. Patent Application No. 61/232,359 entitled “Respiratory Sinus Arrhythmia as a Biometric Indicator”, filed Aug. 7, 2009 are incorporated herein by reference.

Also disclosed herein are methods of predicting a patient's tolerance to a medical event, the method comprising: measuring biometric variables in the patient over time to create a time series data set; and applying a predictive algorithm to the time series.

In some forms, the medical event can include separation from mechanical ventilation. In some forms, the biometric variables can include respiratory variables. In some forms, the medical event can include separation from mechanical ventilation and the biometric variables can include respiratory variables. In some forms the predictive algorithm can be SampEn, ApEn, RRISOD, DisNEn, BPwEN, StatAV or PD2i. In some forms the predictive algorithm can be PD2i. In some forms, a high SampEn, BPWEn and DisNEn values are associated with good ability to of the subject to be removed from the ventilator. In some forms, low SampEn, BPWEn and DisNEn values us associated with poor ability of the subject to be removed from a ventilator, alone or in combination with each other or with other factors. In some forms, the methods can further comprise comparing SampEn, ApEn, RRISOD, DisNEn, BPwEN, StatAV or PD2i values to a standard associated with a particular predictive algorithm. For example, PD2i can be compared to a ventilator removal standard as described elsewhere herein. In another example, SampEN can be compared to a standard based on SampEn data. The SampEn data can, for example, be based on the average SampEn values from subjects that successfully were removed from a ventilator and for subjects that were not successfully removed from the ventilator or from a combination thereof. Similar removal standards can be derived from ApEn, RRISOD, DisNEn, BPwEN, or StatAV values. In some forms, the methods can further comprise recommending removal from a mechanical ventilator if a subjects value is higher or lower than a standard. In some forms the value can be a PD2i, ApEn, RRISOD, DisNEn, BPwEN, or StatAV value. In some forms, the value is higher than the standard. In some forms, the standard is based on the average value from subjects that successfully were removed from a ventilator and for subjects that were not successfully removed from the ventilator or from a combination thereof. In some forms, the respiratory variable include breathing pattern variabilities. In some forms, the algorithm accounts for nonstationarities in the time series data set.

The metrics described herein can be useful predictors of a patient's ability to tolerate separation from mechanical ventilation.

### EXAMPLES

### A. Example 1

### Respiration Interval PD2i

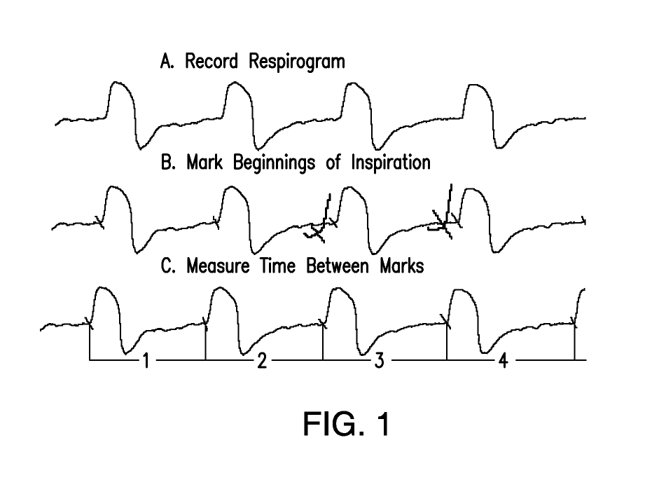

FIG. 1 shows four typical respiration cycles in a respirogram. The digitized respirogram is first made and examined on a computer (A). Then a person or a device can locate the beginning of each inspiration (upward) (B. cross marks); large amplitude visualization can be used (e.g., two inserts) to enable accurate determination of the marks. Then the time interval between the marks is made by counting the number of data points between successive marks (C.). Since each data point has a known time interval, it is then possible to measure the intervals (C. 1 to 4) in real time, to the nearest millisecond (1 integer=1 msec).

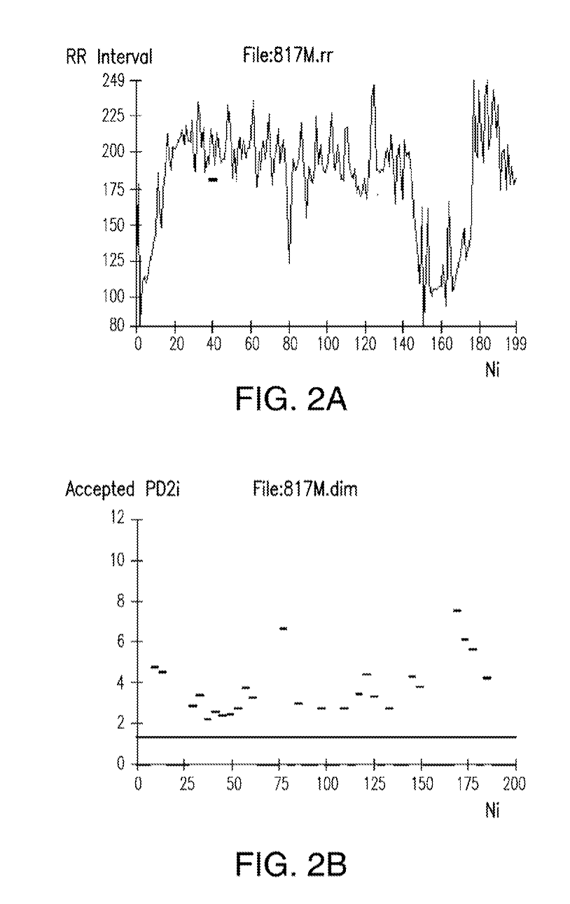

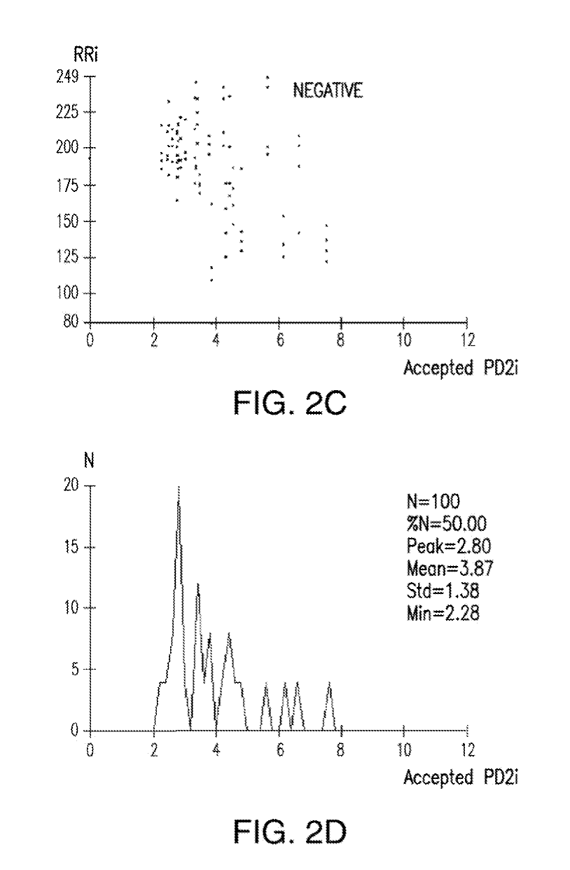

FIG. 2 shows the series of respiratory intervals (A., RR-intervals) and the corresponding PD2i values (B. Accepted PD2i) for each respiratory interval. FIG. 2 C. shows the plot of A. vs B. and 2D. shows the histogram of the accepted PD2i values along with statistics that represent the results. The % N value must be above 30% for a valid determination in data with noise (Skinner, Anchin, Weiss, Therapeutics and Clinical Risk Management, 2008). The outcome for this patients is shown in 2C. (Negative), as the minimum PD2i value is above the cut-point (2.0) found for the entire study.

The M associated with the patient file name (817M) indicates that each respiratory interval in integers (msec) has been modified by a constant reduction in amplitude so as to eliminate noise in the data. This is a common way to reduce noise in physiological data undergoing nonlinear analysis (Skinner patent 2006). In all control and experimental subjects, the respiratory intervals were adjusted to have a mean of 180 integers (reduced by multiplication by approximately 0.25 to 0.125 for most subjects). Integer levels of this size can be shown to reproduce within 4% error the known degrees of freedom (PD2i) in nonstationary calibration data made from sine, Lorenz, Henon and Noise subepochs (Skinner, Molnar, Tomberg, Integrative Physiological and Behavioral Science, 1994).

### B. Example 2

### PD2i Analysis of Respiratory Intervals

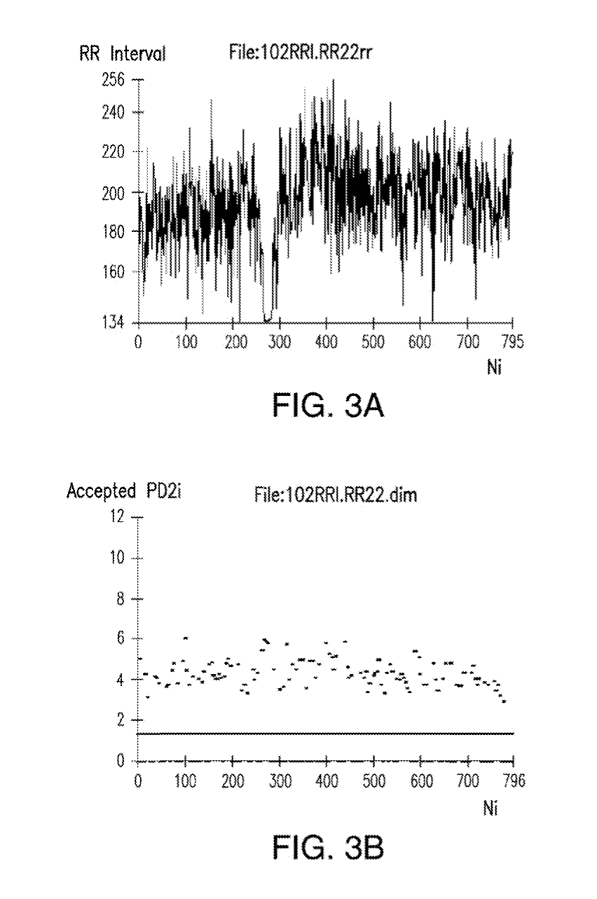

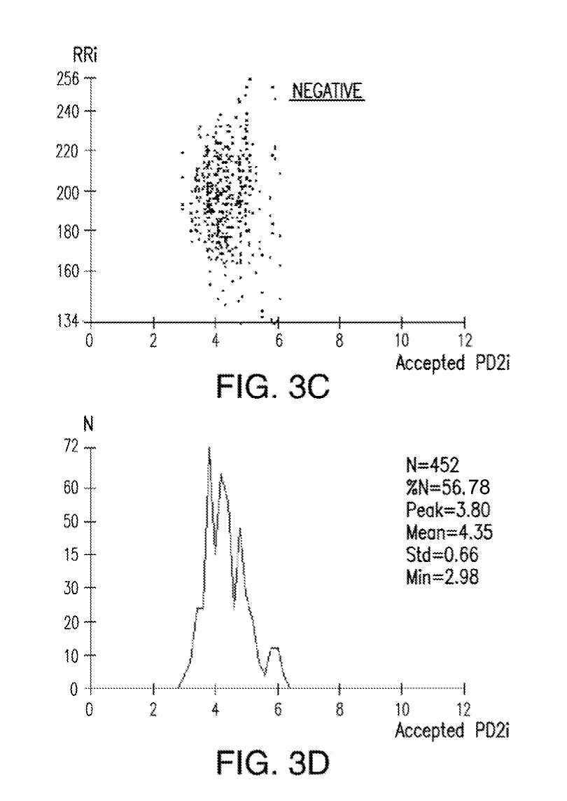

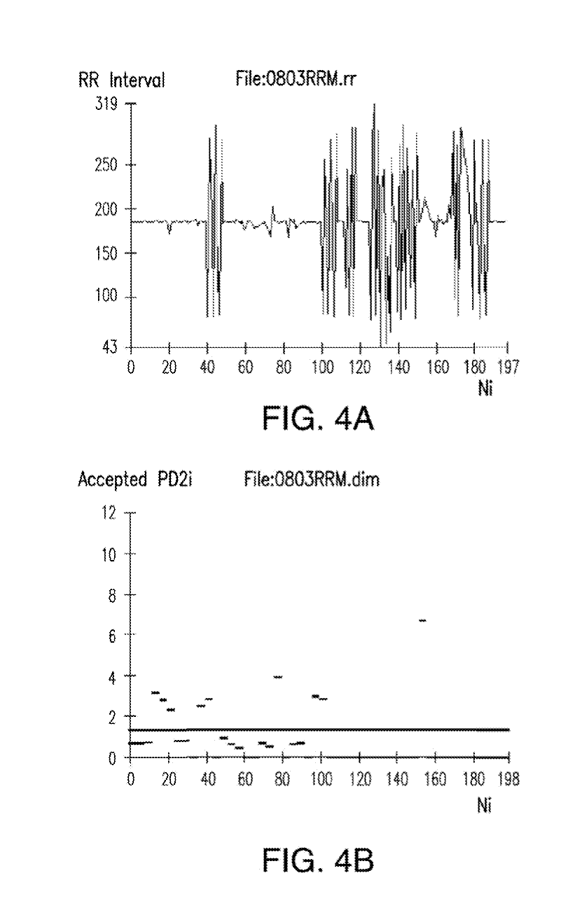

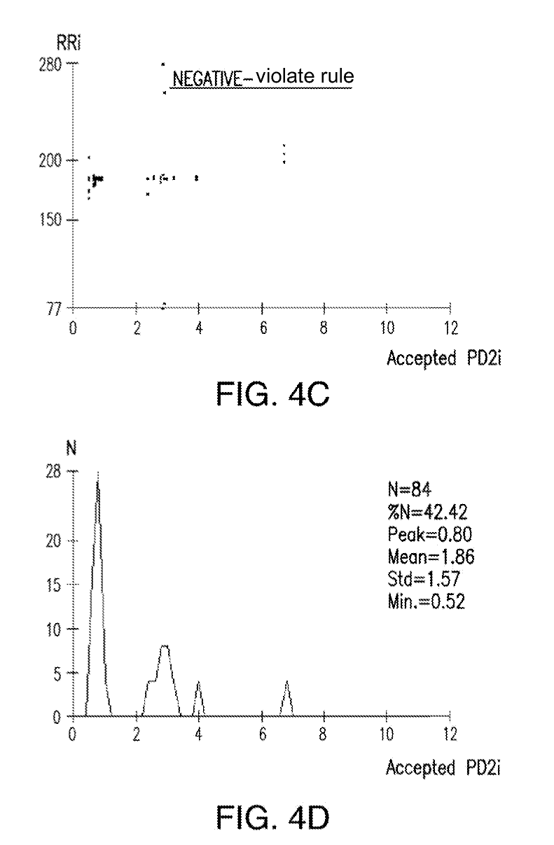

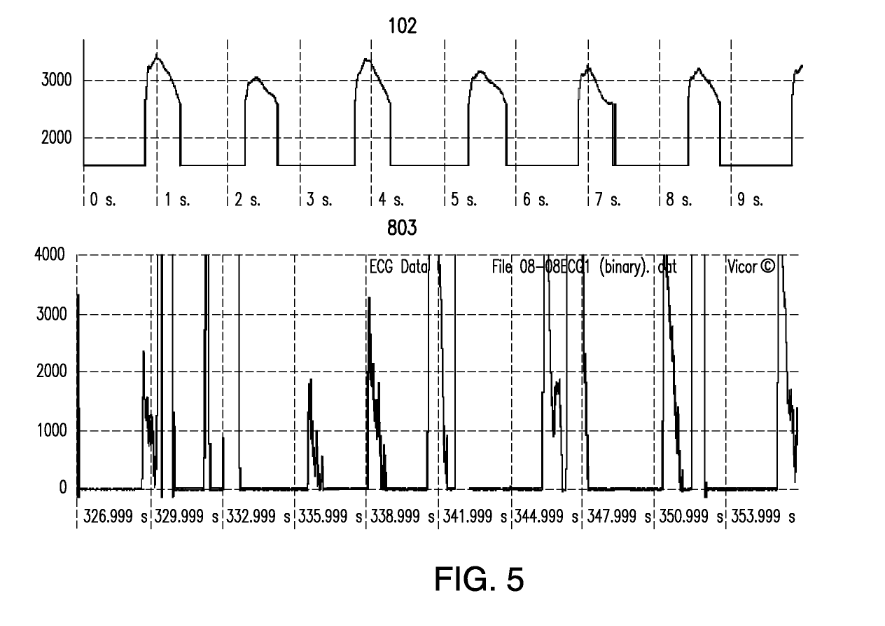

There are qualitative differences in the respiratory mark intervals (RR-like intervals) that do not need statistics to evaluate (see FIG. 3). For both file 102 and 803 the mean respiratory rate was adjusted to be the same (approximately 180 integers). This data set, being expressed in datapoints, has considerably smaller numbers than that for the respiratory rate expressed in time (ms). The amplitude reduction was done to reduce noise in the data so that % N was above 30% (see Skinner, Anchin, Weiss, 2008). So the comparisons were made in modified data with the low-level noise removed by amplitude reduction.

1. Results

The results show statistically significantly lower PD2i values between file 102 (mean PD2i=4.35±0.66 SD) and file 803 (mean PD2i=1.86±1.57 SD), assuming a directional, 1-tailed, null-hypothesis. The Min PD2i values were also significantly different (p<0.026). The data lengths of the two files were different but insignificant. The qualitative differences in respiration are marked (FIG. 3).

2. Conclusions

It is concluded that there are complexity differences in the degrees of freedom between the two files, as measured by the nonlinear PD2i algorithm, when the pattern of respiration is adjusted to the same mean of variation and reduced in amplitude to eliminate noise so as to increase % N scores above the 30% level. The 30% level is required for the physiological data to be statistically significantly different from its randomized phase surrogate (Skinner, Anchin, Weiss, 2008). This surrogate is the same as that of noise with the same power spectrum. For physiological or any other data to be analyzable by a nonlinear algorithm, its algorithmic result must be statistically different from that of noise recorded at the same band-pass (Theiler, 1987).

### C. Example 3

### PD2i Analysis of the Breathing Rate Interval in Patients to be Removed from Ventilators

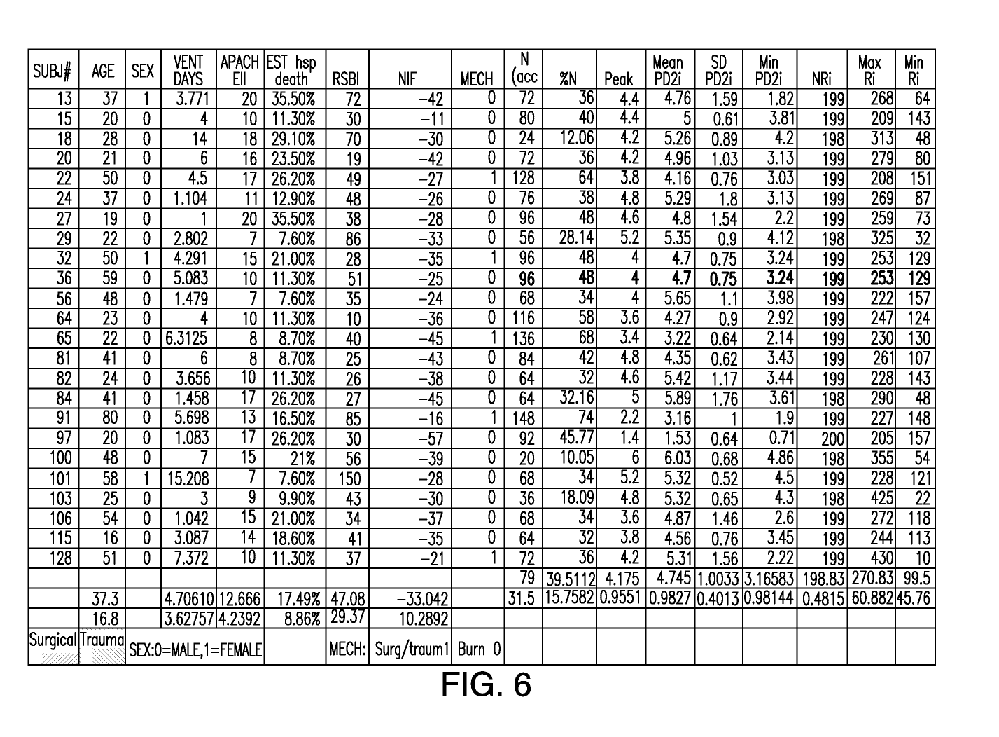

PD2i was used to analyze RRi values of the breathing rate interval in 32 patients between the ages of 16-80. Each patient had been on a ventilator for at least 1 day (FIG. 6). The RRi values were obtained prior to attempting to remove the patients from a ventilator. Each patient was attempted to be removed from a ventilator post obtaining RRi values. The mean Min PD2i value of the patients that were successfully removed from the ventilator was significantly higher compared to the mean Min PD2i value of the patients that could not be removed from the ventilator as determined by having to be placed back on the ventilator quickly, as determined by the attending physician. Statistical analysis of the two mean Min PD2i values showed that the two mean Min PD2i values were statistically significant (t-test, p<0.026).

a) Results

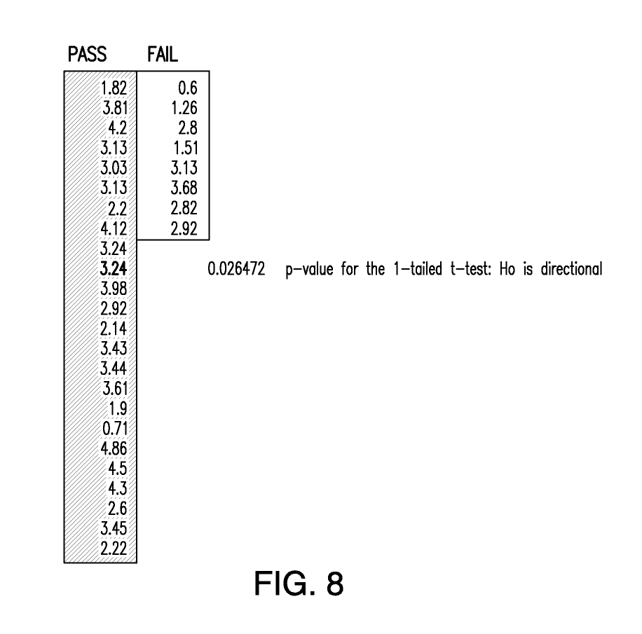

24 of 32 patients were successfully removed from the ventilator, see FIGS. 6 and 8. The mean Min PD2i value for the 24 patients was 3.17 with a standard deviation (SD) of 0.98. The mean Min PD2i value for the 8 patients that could not be removed from the ventilator was 2.34 with a SD of 1.07 (Had to go back on ventilator, as determined by the attending physician). The p-value in a 1-tailed test showed that the two means were statistically different significant with a t-test, p=0.026472, (see FIG. 8).

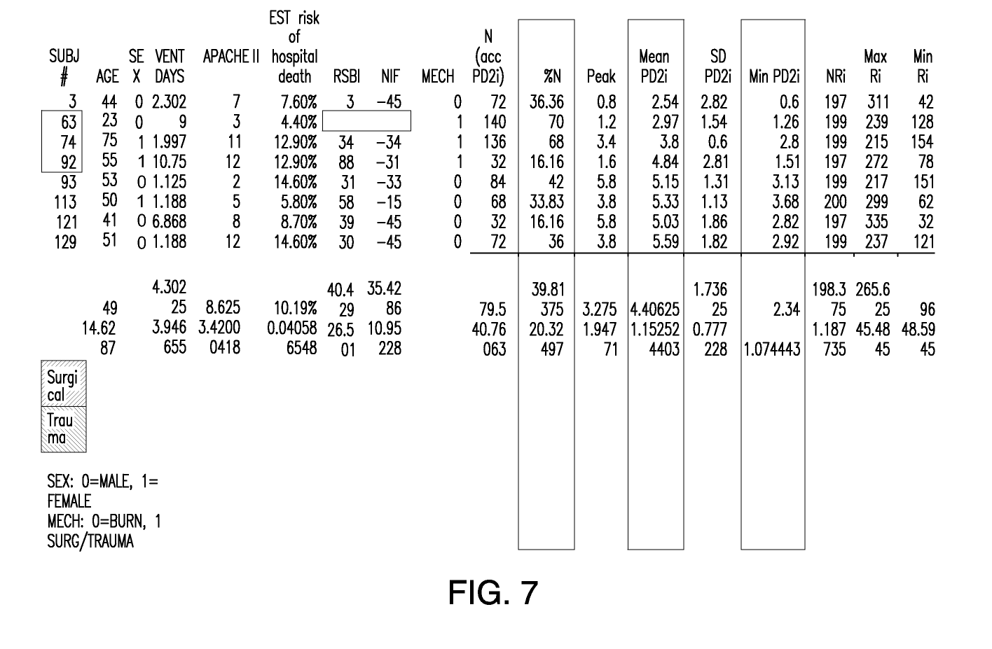

100% of patients with a PD2i value higher than 3.70 were successfully removed from the ventilator. Whereas only 25% of patients with a PD2i value of less then 1.80 were successfully removed from the ventilator. The % N value in FIGS. 6 and 7 denotes all accepted PD2i divided by all possible PD2i; the file can be rejected if less than 30%, however, the data is already reduced in noise by dividing raw respiratory intervals by a number to adjust their means to 180 integers, therefore, the rejections could be neglected, but none were less than 30%.

b) Conclusion

Statistical analysis of the PD2i values shows that patients with higher PD2i values have statistically better chance to be removed from a ventilator. Furthermore, 100% of the patients with a Mean PD2i value higher than 3.70 were successfully removed from the ventilator. While only 25% of the patients with a PD2i value of less than 1.80 were successfully removed from the ventilator. Higher PD2i values can directly be correlated to an increase in probability of successfully removing patients from a ventilator.

### D. Example 4

a) Subjects and Protocol

Appropriate institutional review board approval was obtained prior to the initiation of this study. Because the study was observational and all data were analyzed post-hoc, informed consent was waived. The subjects were prospectively recruited from one Level I trauma center with separate burn and surgical/trauma ICUs during a 9-month period. Both ICUs used an identical SBT protocol. Criteria for inclusion into this study were mechanical ventilation with an endrotracheal tube for >24 hours, regardless of underlying disease, and the ICU attending physician's judgment that the patient was ready for SBT and possible extubation. All SBT were performed with 5 cm H₂O of both positive end-expiratory pressure (PEEP) and pressure support (PS) for 30 minutes. Sedation and analgesia were continued during SBTs at the physician's discretion. The patient was monitored during this time by a respiratory therapist (RT) and returned to the previous ventilator settings if the patient had one or more signs of cardiopulmonary distress listed in table 1.

TABLE 1
Intolerance to SET manifested by:

| | |
|---|---|
| 1 | Significant dyspnea |
| 2 | RR > 39 bpm |
| 3 | Diaphoresis |
| 4 | Use of accessory muscles/thoraco-abdominal paradox |
| 5 | Tachycardia (HR > 120 bpm or increased 20% from baseline) |
| 6 | SBP > 180 OR <90 mm Hg or need for vasopressors |
| 7 | SPO2 < 90% |
| 8 | Change in mental status (coma, confusion, agitation, anxiety) |

If the patient tolerated the SBT, then measurement of respiratory rate (RR), rapid shallow breathing index (RSBI) and negative inspiratory force (NIF) were performed by RT and the physician in charge was contacted and notified of results of SBT. The decision to extubate after “passed” SBT was made by the ICU attending physician. Subjects not extubated after SBT, or subjects re-intubated for elective surgery <48 hours after extubation, were not included in this study. Once extubated, supplemental oxygen was supplied by air-entrapment mask or nasal cannula. Separation from mechanical ventilation was considered a failure if the subject required any ventilatory support, including non-invasive positive pressure ventilation (NPPV), within 48 hours of extubation. Subjects who had undergone separation from mechanical ventilation and failed, or who had passed and were later re-intubated for further surgery, were not considered again for analysis.

b) Waveform Analysis

During the SBT, respiratory flow and pressure waveforms were continuously monitored on the Draeger Evita XLVentilator (Drager Medical, Lubeck, Germany) and the patients were instructed not to speak during the recording period. The waveform data were retrieved from the ventilator for off-line analysis via an RS232 connection recorded at 500 Hz to the DREW digital data acquisition system (Koenig S C, et al., Biomed Instrum Technol 2004; 38:229-240). Recorded data were stored on a personal computer and analyzed by personnel who were blinded to the results of SBT. Two-hundred-breath datasets, which were the most consistently available in all investigated subjects, were imported into WinCPRS software (Absolute Aliens Oy, Turku, Finland). Peaks denoting the beginning of each consecutive respiration were automatically identified by means of an isoelectric line-shift algorithm by the software in every dataset, and correct identification of the all peaks was then manually verified. Both respiratory flow and pressure were used for peak detection to increase the reliability of the process. The software generated the instantaneous inter-breath interval (IBI) time series. Before entropy calculations, linear trends were removed in all segments of the analyzed data. Analysis algorithms are identical to those reported before (Batchinsky A I, et al. J Trauma 2007; 63:512-518; Kuusela T A, et al., Am J Physiol Heart Circ Physiol 2002; 282:H773-783). The following waveform analysis techniques were applied:

1) Approximate entropy (ApEn) and sample entropy (SampEn) measure the amount of irregularity in the R-R interval (RRI) signal (Richman J S, et al., American Journal of Physiology—Heart & Circulatory Physiology 2000; 278:H2039-2049; Kuusela T A, et al., Am J Physiol Heart Circ Physiol 2002; 282:H773-783; Pincus S M. Proc Natl Acad Sci USA 1991; 88:2297-2301). ApEn determines the conditional probability of finding specific patterns in the time series; i.e., the logarithmic likelihood that a run of patterns that is close remains close on the next incremental comparison. The template patterns are constructed from the signal itself, and no a priori knowledge of the system is needed. SampEn is a similar concept to ApEn, with the computational difference that the vector comparison with itself is removed. For both ApEn and SampEn, the dimension parameter m used for calculation was 2 and the filter parameter r was 20% of the standard deviation [see Richman and Moorman (Richman J S, et al., American Journal of Physiology—Heart & Circulatory Physiology 2000; 278:H2039-2049) for discussion of techniques].

2) Similarity of distributions (SOD) explores the probability of similar RRI signal amplitude distributions as a function of time (Zochowski M, et al., Physical Review E 1997; 56:3725-3727).

3) Symbol-dynamics indices: Symbol-distribution entropy (DisNEn) and bit-per-word entropy (BPWEn) collectively measure the probability of patterns within the IBI time series. These metrics are based on recreation of the dynamics of a complex system in phase space. The order in which the dynamics of the system visit the possible encoded regions creates a symbol distribution sequence, DisNEn. Symbol sequences are encoded into words (2 to 3 symbols in length) and the frequency of occurrence of each word is then counted and the normalized entropy (BPWEn) of these words is calculated from a histogram (Hao B. Physica D 1991; 51:161-176).

4) Signal stationarity (StatAv) assesses whether the mean and standard deviation of the signal changes over time during each data set (Palazzolo J A, et al., Am J Physiol 1998; 274:H1099-1105).

c) Statistical Analysis

SAS version 9.1 (SAS Institute, Cary, N.C.) was used for statistical analysis. Normality of continuous variables was assessed with the Shapiro-Wilk test. Univariate analysis was performed with two samples Student's t test or Mann-Whitney U test as appropriate for continuous variables and Fisher's exact test for categorical variables. In addition, Spearman correlation coefficients were calculated to determine relationships between variables. A p value of <0.05 was considered indicative of statistical significance.

d) Results

Thirty-three subjects in this study completed an SBT with 5 cm H₂O PEEP and PS for 30 minutes and were extubated. Of these subjects, one dataset was excluded from analysis because of artifacts in signal. A total of 24 subjects successfully separated from mechanical ventilation. There were eight failures with one failure rescued with NIPPY. The mean duration for time to failure was 22.4 hours (a range of 0.96 to 47.25 hours). There were no deaths in either cohort during the study period. The characteristics of the two groups, along with RR, duration of IBI, NIF and RSBI calculated during SBT, are provided in table 2.

TABLE 2
Group characteristics

| | Pass (N = 24) | Fail (N = 8) | p |
|---|---|---|---|
| Age | 37 ± 17 | 49 ± 15 | 0.08 |
| APACHE II score | 13 ± 4 | 9 ± 3 | 0.02 |
| RR Mean | 30.86 ± 30.12 | 26.15 ± 8.37 | 0.78 |
| NIF | −33 ± 10 | −35 ± 11 | 0.60 |
| RSBI | 47 ± 27 | 40 ± 27 | 0.78 |
| VENT (days) | 4.71 ± 3.63 | 4.30 ± 3.95 | 0.75 |
| Sex (% F) | 13% | 38% | 0.15 |
| MECH (% Surg/Burn) | 21%/79% | 38%/63% | 0.38 |

Mean ± standard deviation; APACHE II, Acute Physiology and Chronic Health, RSBI—Rapid Shallow Breathing Index; NIF—Negative Inspiration Force

Age, sex, and mechanism of injury and duration of mechanical ventilation did not influence outcome and there was no difference in recorded weaning parameters between groups. However, the Acute Physiology and Chronic Health Evaluation (APACHE) II score on admission was higher in the success group (p<0.05).

Nonlinear results are provided in table 3. As measured by SampEn the IBI in the success group was more irregular than in the failure group, in which the subjects had a lower SampEn and thus a more regular IBI distribution. ApEn, however, was not different between groups. SOD was lower in the success group, implying a more dissimilar signal distribution; and higher in the failure group, pointing to a more regular signal amplitude distribution. The stationarity value (StatAv), which measures baseline shifts in the signal, was not different among groups (see below for discussion on this metric). BPWEn and DisNEn changed in concordance with SampEn and denoted lower signal irregularity in the failure group (table 3). Last, there was no correlation between SampEn value and time to failure.

TABLE 3
Non-linear results

| | Pass (N = 24) | Fail (N = 8) | P |
|---|---|---|---|
| SampEn | 1.87 ± 0.27 | 1.36 ± 0.39 | 0.00 |
| ApEn | 0.97 ± 0.06 | 0.93 ± 0.11 | 0.36 |
| RRISOD | 0.17 ± 0.03 | 0.24 ± 0.05 | 0.01 |
| DisNEn | 0.82 ± 0.06 | 0.75 ± 0.06 | 0.01 |
| BPwEN | 4.94 ± 0.38 | 4.51 ± 0.34 | 0.01 |
| StatAv | 0.33 ± 0.13 | 0.29 ± 0.10 | 0.88 |

Mean ± standard deviation

e) Discussion

The primary finding of this study is that in intubated patients undergoing SBT, the IBIs in those who failed to separate from mechanical ventilation were more regular than in those who were successfully extubated. This implies a lower regulatory complexity of respiration as measured by different nonlinear methods. As collective measures of regulatory complexity, these methods can then be useful markers in predicting outcome of SBT when available at bedside. Also, RR, NIF and RSBI did not differ between groups and that all subjects who were extubated had weaning parameters predictive of success.

Different statistical techniques were used to determine the complexity of the respiratory signal. First, entropy metrics (ApEn, SampEn, DisNen and BPWEn) were used to measures the amount of irregularity in the signal. Both ApEn and SampEn calculate the (logarithmic) likelihood that clusters of patterns that are close in time remain close in the next incremental comparison; that is, how knowing one portion of the signal will allow forecasting of the next portion as it is moved forward in time. They are nonlinear metrics that are scale- and model-independent and produce non-negative numbers that can be used for comparisons across studies; a higher number represents higher irregularity. SampEn differs from ApEn by disallowing self-matches and appears more robust, as SampEn can provide meaningful clinical results using datasets as short as 100 beats in length (Richman J S, et al., American Journal of Physiology—Heart & Circulatory Physiology 2000; 278:H2039-2049, Pincus S M. Proc Natl Acad Sci USA 1991; 88:2297-2301). SampEn calculated for the two groups presented in this study was different with the cohort that failed extubation having a mean value lower (1.35+/−0.39 vs. 1.87+/−0.27; p<0.001), although ApEn was not different (0.93+/−0.11 vs. 0.97+/−0.06, fail vs. success, respectively; p=0.36). DisNEn and BPWEn tend to move in concert with SampEn and all were lower in the failure group. These former two measures represent the signal distribution in phase space and, albeit methodologically distinct from SampEn, are complementary entropy measures of signal irregularity. Similarly to this study, changes in DisNEn and BPWEn have followed the trend in SampEn in previous studies during hemorrhagic shock in animals (Batchinsky A I, et al., Crit Care Med 2007; 35:519-525) as well as burn shock in humans (Batchinsky A I, et al., Journal of Burn Care and Research 2008; 29:56-63).

Another technique used, SOD, converts the signal into histograms (amplitude distributions) that are set in arbitrary time windows and then explores the probability that similar histograms will recur as a function of time. SOD is indirectly related to complexity and is scored as a probability between 0 (no recurrence) and 1 (complete overlap of histograms). It is also robust in signal analysis and can provide meaningful results in small datasets (Batchinsky A I, et al., Shock 2009). In this study, SOD was higher in the cohort that failed extubation (0.23+/−0.05 vs. 0.17+/−0.03, respectively; p<0.02).

Ectopic beats that occur during EKG recording or coughing with respiratory recordings can create noise and errors during signal analysis. These nonstationary signals are identified by changes in the mean and standard deviation of the signal during the course of a dataset. Both SampEn and SOD are generally more robust to nonstationarities in patient data than other metrics; the effect of noise on SampEn is predictable, causing a slightly greater value. Assessment of the signal quality used for the above comparisons was tested by means of StatAv. This metric assesses baseline shifts in means and standard deviations over the time course of the dataset and is higher in less stationary signals. StatAv was low, pointing to low signal nonstationarity, and was also similar between the two groups (0.33+/−0.13 vs. 0.30+/−0.10, failure vs. success, respectively; p=0.88), which indicates equal effects of data quality on the metrics in both groups.

The pulmonary system is a biological, nonlinear system characterized by the rhythmic activity of respiratory central pattern generators. The respiratory pattern in healthy, awake adults is characterized by breath-to-breath variability in the rate, duration and size of breaths (El-Khatib M, et al., Intensive Care Medicine 2001; 27:52-58; Engoren M. Critical Care Medicine 1998; 26:1817-1823; Bien M Y, et al., Intensive Care Medicine 2004; 30:241-247; Wysocki M, et al., Critical Care Medicine 2006; 34:2076-2083). This variability is not purely random but rather is a manifestation of long-range correlations that exist among the fluctuations in one or more respiratory variables extending over hundreds of breathing cycles (Wysocki M, et al., Critical Care Medicine 2006; 34:2076-2083; Fadel P J, et al., Journal of Applied Physiology 2004; 97:2056-2064). The respiratory system therefore has a “memory effect” such that the value of a present measured variable is related to those in the distant past. This effect also appears to extend over more than one time scale, which may indicate different levels of network control (Fadel P J, et al., Journal of Applied Physiology 2004; 97:2056-2064; Gebber G L, et al., Conference Proceedings: Annual International Conference of the IEEE Engineering in Medicine & Biology Society 2006; 1:4615-4618). These long-range correlations point to the fractal organization of human physiologic breathing. A fractal is a structure that is self-similar and is time and scale invariant such that shorter sections are similar in structure to longer sections (Mandlebrot B. The fractal geometry of nature. New York: Freeman; 1982). This memory effect can be a product of the self-similar nature of the respiratory signal (Goldberger A L. Lancet 1996; 347:1312-1314). A signal that is more fractal in nature is more complex and more richly regulated and, as a result of long-range correlations, may have some predictive value in modeling future behavior of the system (Goldberger A L. Lancet 1996; 347:1312-1314; Goldberger A L., Yale Journal of Biology & Medicine 1987; 60:421-435). However, since nonlinear systems exhibit sensitivity to initial conditions, accurate long range predictions are not possible (Goldberger A L., Yale Journal of Biology & Medicine 1987; 60:421-435; Williams G P. Choas Theory Tamed. Washington, D.C.: Joseph Henry Press; 1997). This factor may be the reason that there was no correlation between SampEn of those who failed and time to failure.

The respiratory center resides in the brainstem and integrates input from multiple areas to including both central and peripheral chemoreceptors, chest wall and muscle mechanoreceptors, pulmonary receptors, vagal afferents, the cerebrum and other central non-respiratory centers (Engoren M. Critical Care Medicine 1998; 26:1817-1823; Cunningham D J, et al., Journal of Physiology 1986; 376:31-45; Bruce E N, et al., Journal of Applied Physiology 1987; 62:389-402; Caruana-Montaldo B, et al., Chest 2000; 117:205-225; Fink B R. Journal of Applied Physiology 1961; 16:15-20; Bianchi A L, et al., Physiological Reviews 1995; 75:1-45). The respiratory pattern is a nonlinear, dynamic output signal that is a consequence of these mutual interactions and the structural complexity of this signal may be a reflection of the regulatory complexity of its control system. In fact, a principal hypothesis in complexity theory holds that system stability “depends on the number, bias and types of interconnections among the system's constituents (Godin P J, et al., Critical Care Medicine 1996; 24:1107-1116).” Conversely, greater signal regularity may be a surrogate for system isolation, or “decomplexification” in nonlinear systems; and multiple system organ failure may be a consequence this loss of coupling between communicating organ systems (; Pincus S M. Mathematical Biosciences 1994; 122:161-181, Goldberger A L. Lancet 1996; 347:1312-1314; Buchman T G, Nature 2002; 420:246-251). In these cases, loss of signal complexity may be a result of a relaxation of feedback mechanism and reveal more simple control of the system or an adaptive strategy in times of stress (Godin P J, et al., Critical Care Medicine 1996; 24:1107-1116; Buchman T G, Nature 2002; 420:246-251). This has been extensively studied in the heart where decreased variability of RRI was associated with disease states as well as aging (Rassias A J, et al., Critical Care Medicine 2005; 33:512-519; Cancio L C, et al., Journal of Trauma-Injury Infection & Critical Care 2008; 65:813-819; Kaplan D T, et al., Biophysical Journal 1991; 59:945-949; Singer D H, et al., Journal of Electrocardiology 1988; 21 Suppl:S46-55; Hogue C W, et al., Circulation 1998; 98:429-434). In hemorrhage and/or shock models, resuscitation is associated with a progressive increase in RRI variability (Batchinsky A I, et al., Crit Care Med 2007; 35:519-525; Batchinsky A I, et al., Journal of Burn Care and Research 2008; 29:56-63).

In the respiratory system, loss of variability also occurs in healthy human volunteers, where adding elastic or resistive loads (Brack T, et al., American Journal of Respiratory & Critical Care Medicine 1998; 157:1756-1763), or challenge with endotoxin (Preas H L, et al., American Journal of Respiratory & Critical Care Medicine 2001; 164:620-626), decreased breath-to-breath variability. It is reduced during sleep and also degrades with age (Peng C K, et al., Annals of Biomedical Engineering 2002; 30:683-692; Modarreszadeh M, et al., Journal of Applied Physiology 1990; 69:630-639). In disease states such as restrictive or obstructive pulmonary disease, patients adopt more constrained breathing patterns (Brack T, et al., American Journal of Respiratory & Critical Care Medicine 2002; 165:1260-1264, Loveridge B, et al., American Review of Respiratory Disease 1984; 130:730-733). Under stress, the frequency to tidal volume ratio increases and both tidal volume and respiratory period become more monotonic. This adaptive strategy is more energy-efficient since smaller breaths are less costly than one breath twice as large (Marini J J, et al., Critical Care Medicine 2006; 34:2241-2243). However, in patients who fail weaning trails, this rapid shallow breathing patterns occurs immediately after discontinuation of mechanical ventilation (Tobin M J, et al., American Review of Respiratory Disease 1986; 134:1111-1118) and is also manifest simultaneously in the electromyographic power spectrum of the diaphragmatic muscles by changes in the ratio of high-to-low frequency power (Cohen C A, et al, American Journal of Medicine 1982; 73:308-316; Brochard L, et al., American Review of Respiratory Disease 1989; 139:513-521). Assessed along two dimensions, respiratory sinus arrhythmia (RSA), which couples heart rate-variability with respiration, is attenuated with hypoxia but strengthened by hypercarbia (Yasuma F, et al., Chest 2004; 125:683-690). Moreover, “programming” variability into mechanical ventilators (i.e., fractal ventilation) improves gas exchange in animal models, which may be the result of increased recruitment of collapsed alveoli with nonlinear opening characteristics or perhaps stronger coupling between nonlinear biological oscillators or both (Boker A, et al. American Journal of Respiratory & Critical Care Medicine 2002; 165:456-462; Mutch W A, et al., American Journal of Respiratory & Critical Care Medicine 2000; 162:319-323; Suki B, et al., Nature 1998; 393:127-128).

In this current study, the proxy for improving respiratory health was successful extubation, and these patients demonstrated more irregular (i.e., more complex) breathing patterns than those who failed. Consequently, complexity of breathing patterns may be a manifestation of an improved pulmonary load balance through increased respiratory reserve. If this is the case, then the appropriateness of therapeutic interventions (i.e., antibiotics) may be marked by increasing complexity in measured pulmonary variables. Alternatively, changes in complexity of the respiratory pattern over time may cause changes in the load capacity balance faced by the pulmonary system; in this case, increasing complexity of breathing patterns may result in increased functional reserve capacity through decreased atelectasis for the reasons mentioned above for fractal ventilation. Neither is mutually exclusive, and we hypothesize that both are involved and in fact may represent hierarchies of control: Locally, increasing complexity of breathing patterns improves load balance within the pulmonary system; globally, increasing connectivity between organs and the central respiratory controller increases signal complexity output to the respiratory system. Because long-range connections between organ systems require time to re-form, initially then, local control may play the larger role in increasing respiratory signal complexity through feedback mechanisms (Kauffman S A, Johnsen S. Journal of Theoretical Biology 1991; 149:467-505).

Wysoki and colleagues compared 51 consecutive patients who were mechanically ventilated >24 hours and measured multiple respiratory variables while undergoing an hour long SBT (Wysocki M, et al., Critical Care Medicine 2006; 34:2076-2083). In this study, patients were disconnected from the ventilator and received only supplemental oxygen during the SBT. The recordings were stratified into success and failure to remain free of ventilatory support for >48 hours (those who were reconnected to the ventilator during or at the end of the SBT were considered failed trials), and coefficients of variation (CV=standard deviation expressed as a percentage of the mean) derived from data. All CVs of the respiratory variables were higher in the patients who successfully separated from the ventilator than in the subjects who failed. These results are consistent with Bien and colleagues' finding in which in which 78 mechanically ventilated systemic inflammatory response syndrome (SIRS) patients who had undergone abdominal surgery were studied for 30 minutes during a SBT while receiving 5 cm H₂O pressure support (Bien M Y, et al., Intensive Care Medicine 2004; 30:241-247). The CV of five respiratory variables were lower in the failure group than in those who successfully extubated. Both studies are in line with our data that increasing breathing variability predicted successful separation from mechanical ventilation.

El-Khatib and colleagues studied 52 intubated patients for variability in tidal volume (V_{T}) and peak inspiratory flow during synchronized mechanical ventilation (rate ≦4 breaths/minute) followed by continuous positive airway pressure (CPAP) trials and showed that increased variability in both variables was associated with extubation failure (El-Khatib M, et al., Intensive Care Medicine 2001; 27:52-58). The majority of the patients in this latter study had underlying lung disease and required a longer duration of ventilator support. For this study, failure was defined as re-intubation within 24 hours not caused by stridor. Of note, four patients in our study failed after 24 hours, with none requiring re-incubation beyond 48 hours; one was re-incubated for stridor. Although this current study did not examine these variables, it is different from our hypothesis that variability is associated with improving respiratory health. In fact, these results are in contrast with the two former studies in which the CV of V_{T} of both success groups was similar (25% and 28%, respectively)(Bien M Y, et al., Intensive Care Medicine 2004; 30:241-247; Wysocki M, et al., Critical Care Medicine 2006; 34:2076-2083) and also in line with the normal range of tidal variation reported in the literature (10, 21, 36); however, the CV for V_{T} in El-Khatib and colleagues success group was 9% (El-Khatib M, et al., Intensive Care Medicine 2001; 27:52-58).

Using ApEn, Engoren investigated the regularity of RR and V_{T} signals in three groups of post cardiac surgery patients (Engoren M. Critical Care Medicine 1998; 26:1817-1823). The first group was studied within 12 hours of surgery and underwent SBT with 5 cm H₂O continuous positive airway pressure; all were extubated successfully and served as the control group. The second and third groups consisted of 21 patients who were mechanically ventilated ≧7 days and underwent 60-to-120 minute SBT with 5 cm H₂O PEEP and various levels of PS. These were then stratified into success versus failure to wean (with or without extubation), and many subjects contributed more than one weaning attempt to the analysis. In this study, although V_{T} did not vary between groups, its ApEn was highest in those who failed weaning trials, with increasing RR across groups having no effect on pattern. These results are in contrast to recent studies (Bien M Y, et al., Intensive Care Medicine 2004; 30:241-247; Wysocki M, et al., Critical Care Medicine 2006; 34:2076-2083). The two experimental groups presented by Engoren were ventilator-dependent at the time of the SBT, which were subsequently conducted for 60 to 120 minutes with 5 cm H₂O of PEEP and higher levels of PS. In fact, those with the highest variability were supported with a mean of 12.2+/−4.6 cm H₂O of pressure support. However, the use of PS should reduce V_{T} variability because the pressure remains the same for all breaths (Brochard L. Critical Care Medicine 1998; 26:1773-1774). Caminal and colleagues have shown an indirect relationship between PS and CV of V_{T}, T_{I} and total breath duration (Wysocki M, et al., Critical Care Medicine 2006; 34:2076-2083; Caminal P, et al., Medical & Biological Engineering & Computing 2004; 42:86-91). This reflects the unloading of the respiratory system by the ventilator and results in breathing patterns that are more characteristic of the ventilator/patient interface than the patient's own intrinsic rhythm (Wysocki M, et al., Critical Care Medicine 2006; 34:2076-2083; Brochard L. Critical Care Medicine 1998; 26:1773-1774); and this highlights the need to assess “prevailing conditions” (i.e., underlying disease, level of ventilator support, mental status, secretions, drugs, fever, etc.) when studying respiratory variability (Wysocki M, et al., Critical Care Medicine 2006; 34:2076-2083). Likewise, it may also explain the conflicting data on respiratory variables given the longer duration of mechanical ventilation in some studies.

This current study was performed at one Level I trauma center with separate burn and surgical/trauma ICUs. For logistic reasons, more burn patients were enrolled in this study; therefore, the results presented here may not be applicable to other patient populations and need to be validated in a larger, more diverse cohort. A second limitation of this study was that sedation and analgesia were not strictly controlled during the SBT but were left to the attending physician's judgment. General anesthesia has been shown to reduce breathing variability (Wysocki M, et al., Critical Care Medicine 2006; 34:2076-2083, Sammon M P, et al., Journal of Applied Physiology 1991; 70:1748-1762), and propofol may cause rapid shallow breathing if continued during an SBT (Khamiees M, et al., Respiratory Care 2002; 47:150-153); both benzodiazepines and narcotics depress the respiratory drive, and other drugs (e.g., beta-blockers, alpha-adrenergics), given at time of an SBT, may affect measured respiratory pattern. Since it has been demonstrated that the respiratory pattern may “speed up” or “slow down” without changing entropy measures (Engoren M. Critical Care Medicine 1998; 26:1817-1823), it is not clear what effect these drugs have on respiratory signal regularity. However, enrollment in this study was made at the attending physician's judgment that the patient was ready for the SBT and possible extubation. All SBTs were done by protocol, with 5 cm H₂O PEEP and PS for 30 minutes, which had been established across ICUs at our institution before initiation of the study. The decision to extubate was made at the end of the SBT by the attending physician, and no patient required re-intubation beyond 48 hours, a time point also chosen in two recent studies (Bien M Y, et al., Intensive Care Medicine 2004; 30:241-247; Wysocki M, et al., Critical Care Medicine 2006; 34:2076-2083).

The IBI was examined with complexity metrics because previous work demonstrated the fractal organization of this respiratory variable (C K, Mietus J et al., Annals of Biomedical Engineering 2002; 30:683-692; Fadel P J, et al., Journal of Applied Physiology 2004; 97:2056-2064) and that the central respiratory controller (rhythm generating function) was more constant than its drive components (Tobin M J, et al., Journal of Applied Physiology 1988; 65:309-317). The use of SampEn has been extensively studied and validated in the cardiac system and was conducted here according to those methodologies. The SOD has complemented the results of SampEn in recent RRI studies (Richman J S, et al., American Journal of Physiology—Heart & Circulatory Physiology 2000; 278:H2039-2049; Batchinsky A I, Shock 2009; Kuusela T A, et al., Am J Physiol Heart Circ Physiol 2002; 282:H773-783; Batchinsky A I, et al., Journal of Burn Care and Research 2008; 29:56-63). One dataset was removed from analysis as a result of artifacts which made it impossible to analyze. Of the remaining datasets, 200 breaths of recordings were compared in toto (i.e., the signal was not edited and there were no discontinuities within datasets) from both success and failure groups for calculation of SampEn and SOD; therefore, phasing between datasets remained true.

Overall, lower SampEn, BPWEn and DisNEn and higher SOD of IBIs were associated with extubation failure. These findings indicate a lower regulatory complexity of respiration as measured by these metrics.

### REFERENCES

1. Rothaar R, Epstein S. Extubation failure: magnitude of the problem, impact on outcomes and prevention. Current Topics in Critical Care 2003; 9:56-66.
2. Epstein S, Ciubotaru R, Wong J. Effect of failed extubation on the outcome of mechanical ventilation. Chest 1997; 112:186-192.
3. Tobin M J, Perez W, Guenther S M, et al. The pattern of breathing during successful and unsuccessful trials of weaning from mechanical ventilation. American Review of Respiratory Disease 1986; 134:1111-1118.
4. Kollef M, Shapiro S, Silver P. A randomized, controlled trial of protocol directed versus physician directed weaning from mechanical ventilation. Critical Care Medicine 1997; 25:567-574.
5. Meade M, Guyatt G, Cook D. Predicting success in weaning from mechanical ventilation. Chest 2001; 120:4005-424S.
6. El-Khatib M, Jamaleddine G, Soubra R, et al. Pattern of spontaneous breathing: potential marker for weaning outcome. Spontaneous breathing pattern and weaning from mechanical ventilation. Intensive Care Medicine 2001; 27:52-58.
7. Engoren M. Approximate entropy of respiratory rate and tidal volume during weaning from mechanical ventilation.[see comment]. Critical Care Medicine 1998; 26:1817-1823.
8. Bien M Y, Hseu S S, Yien H W, et al. Breathing pattern variability: a weaning predictor in postoperative patients recovering from systemic inflammatory response syndrome. Intensive Care Medicine 2004; 30:241-247.
9. Wysocki M, Cracco C, Teixeira A, et al. Reduced breathing variability as a predictor of unsuccessful patient separation from mechanical ventilation.[see comment]. Critical Care Medicine 2006; 34:2076-2083.
10. Tobin M J, Mador M J, Guenther S M, et al. Variability of resting respiratory drive and timing in healthy subjects. Journal of Applied Physiology 1988; 65:309-317.
11. Benchetrit G. Breathing pattern in humans: diversity and individuality. Respiration Physiology 2000; 122:123-129.
12. Peng C K, Mietus J E, Liu Y, et al. Quantifying fractal dynamics of human respiration: age and gender effects. Annals of Biomedical Engineering 2002; 30:683-692.
13. Brack T, Jubran A, Tobin M J, et al. Dyspnea and decreased variability of breathing in patients with restrictive lung disease. American Journal of Respiratory & Critical Care Medicine 2002; 165:1260-1264.
14. Leigh R, Shaw D. Rapid regular respiration in unconscious patients. Archives of Neurology 1976; 33:356-361.
15. Loveridge B, West P, Anthonisen N R, et al. Breathing patterns in patients with chronic obstructive pulmonary disease. American Review of Respiratory Disease 1984; 130:730-733.
16. Brack T, Jubran A, Tobin M J. Effect of resistive loading on variational activity of breathing. American Journal of Respiratory & Critical Care Medicine 1998; 157:1756-1763.
17. Preas H L, 2nd, Jubran A, Vandivier R W, et al. Effect of endotoxin on ventilation and breath variability: role of cyclooxygenase pathway. American Journal of Respiratory & Critical Care Medicine 2001; 164:620-626.
18. Jubran A, Tobin M. Effect of isocapnic hypoxia on variational activity of breathing. American Journal of Respiratory & Critical Care Medicine 2000; 162:1202-1209.
19. Jubran A, Grant B, Tobin M. Effect of hyperoxic hypercapnea on variational activity of breathing. American Journal of Respiratory & Critical Care Medicine 1997; 156:1129-1139.
20. Brack T, Jubran A, Tobin M. Effect of elastic loading on variational activity of breathing. American Journal of Respiratory & Critical Care Medicine 1997; 155:1341-1348.
21. Shore E, Millman R, Silage D. Ventilatory and arousal patterns during sleep in normal young and elderly subjects. Journal of Applied Physiology 1984; 59:1605-1615.
22. Gilbert R, Auchincloss J, Peppi D, et al. The first few hours off a respirator. Chest 1974; 65:152-157.
23. Godin P J, Buchman T G. Uncoupling of biological oscillators: a complementary hypothesis concerning the pathogenesis of multiple organ dysfunction syndrome. Critical Care Medicine 1996; 24:1107-1116.
24. Pincus S M. Greater signal regularity may indicate increased system isolation. Mathematical Biosciences 1994; 122:161-181.
25. Batchinsky A I, Cancio L C, Salinas J, et al. Prehospital loss of R-to-R interval complexity is associated with mortality in trauma patients. J Trauma 2007; 63:512-518.
26. Richman J S, Moorman J R. Physiological time-series analysis using approximate entropy and sample entropy. American Journal of Physiology—Heart & Circulatory Physiology 2000; 278:H2039-2049.
27. Batchinsky A I, Salinas J, Tom K, et al. Rapid prediction of trauma-patient survival by analysis of heart-rate complexity: impact of reducing dataset size. Shock 2009; In press.
28. Zochowski M, Winkowska-Nowak K, Nowak A, et al. Autocorrelations of R-R distributions as a measure of heart variability. Physical Review E 1997; 56:3725-3727.
29. Hao B. Symbolic dynamics and characterization of complexity. Physica D 1991; 51:161-176.
30. Palazzolo J A, Estafanous F G, Murray P A. Entropy measures of heart rate variation in conscious dogs. Am J Physiol 1998; 274:H1099-1105.
31. Koenig S C, Woolard C, Drew G, et al. Integrated data acquisition system for medical device testing and physiology research in compliance with good laboratory practices. Biomed Instrum Technol 2004; 38:229-240.
32. Kuusela T A, Jartti T T, Tahvanainen K U, et al. Nonlinear methods of biosignal analysis in assessing terbutaline-induced heart rate and blood pressure changes. Am J Physiol Heart Circ Physiol 2002; 282:H773-783.
33. Pincus S M. Approximate entropy as a measure of system complexity. Proc Natl Acad Sci USA 1991; 88:2297-2301.
34. Batchinsky A I, Cooke W H, Kuusela T, et al. Loss of complexity characterizes the heart-rate response to experimental hemorrhagic shock in swine. Crit Care Med 2007; 35:519-525.
35. Batchinsky A I, Wolf S E, Molter N, et al. Assessment of cardiovascular regulation after burns by nonlinear analysis of the electrocardiogram. Journal of Burn Care and Research 2008; 29:56-63.
36. Fadel P J, Barman S M, Phillips S W, et al. Fractal fluctuations in human respiration. Journal of Applied Physiology 2004; 97:2056-2064.
37. Gebber G L, Barman S M, Fadel P J, et al. Fractal fluctuations in breath number, period, and amplitude are independently controlled in awake, healthy humans. Conference Proceedings: Annual International Conference of the IEEE Engineering in Medicine & Biology Society 2006; 1:4615-4618.
38. Mandlebrot B. The fractal geometry of nature. New York: Freeman; 1982.
39. Goldberger A L. Non-linear dynamics for clinicians: chaos theory, fractals, and complexity at the bedside. Lancet 1996; 347:1312-1314.
40. Goldberger A L, West B J. Fractals in physiology and medicine. Yale Journal of Biology & Medicine 1987; 60:421-435.
41. Williams G P. Choas Theory Tamed. Washington, D.C.: Joseph Henry Press; 1997.
42. Cunningham D J, Howson M G, Metias E F, et al. Patterns of breathing in response to alternating patterns of alveolar carbon dioxide pressures in man. Journal of Physiology 1986; 376:31-45.
43. Bruce E N, Cherniack N S. Central chemoreceptors. Journal of Applied Physiology 1987; 62:389-402.
44. Caruana-Montaldo B, Gleeson K, Zwillich C W. The control of breathing in clinical practice. Chest 2000; 117:205-225.
45. Fink B R. Influence of cerebral activity in wakefulness on regulation of breathing. Journal of Applied Physiology 1961; 16:15-20.
46. Bianchi A L, Denavit-Saubie M, Champagnat J. Central control of breathing in mammals: neuronal circuitry, membrane properties, and neurotransmitters. Physiological Reviews 1995; 75:1-45.
47. Buchman T G, Buchman T G. The community of the self. Nature 2002; 420:246-251.
48. Rassias A J, Holzberger P T, Givan A L, et al. Decreased physiologic variability as a generalized response to human endotoxemia.[see comment]. Critical Care Medicine 2005; 33:512-519.
49. Cancio L C, Batchinsky A I, Salinas J, et al. Heart-rate complexity for prediction of prehospital lifesaving interventions in trauma patients. Journal of Trauma-Injury Infection & Critical Care 2008; 65:813-819.
50. Kaplan D T, Furman M I, Pincus S M, et al. Aging and the complexity of cardiovascular dynamics. Biophysical Journal 1991; 59:945-949.
51. Singer D H, Martin G J, Magid N, et al. Low heart rate variability and sudden cardiac death. Journal of Electrocardiology 1988; 21 Suppl:S46-55.
52. Hogue C W, Jr., Domitrovich P P, Stein P K, et al. RR interval dynamics before atrial fibrillation in patients after coronary artery bypass graft surgery.[see comment]. Circulation 1998; 98:429-434.
53. Modarreszadeh M, Bruce E N, Gothe B. Nonrandom variability in respiratory cycle parameters of humans during stage 2 sleep. Journal of Applied Physiology 1990; 69:630-639.
54. Marini J J, Marini J J. Breathing patterns as integrative weaning predictors: Variations on a theme. Critical Care Medicine 2006; 34:2241-2243.
55. Cohen C A, Zagelbaum G, Gross D, et al. Clinical manifestations of inspiratory muscle fatigue. American Journal of Medicine 1982; 73:308-316.
56. Brochard L, Harf A, Lorino H, et al. Inspiratory pressure support prevents diaphragmatic fatigue during weaning from mechanical ventilation.[see comment]. American Review of Respiratory Disease 1989; 139:513-521.
57. Yasuma F, Hayano J, Yasuma F, et al. Respiratory sinus arrhythmia: why does the heartbeat synchronize with respiratory rhythm?[see comment]. Chest 2004; 125:683-690.
58. Boker A, Graham M R, Walley K R, et al. Improved arterial oxygenation with biologically variable or fractal ventilation using low tidal volumes in a porcine model of acute respiratory distress syndrome. American Journal of Respiratory & Critical Care Medicine 2002; 165:456-462.
59. Mutch W A, Harms S, Ruth Graham M, et al. Biologically variable or naturally noisy mechanical ventilation recruits atelectatic lung.[see comment]. American Journal of Respiratory & Critical Care Medicine 2000; 162:319-323.
60. Suki B, Alencar A M, Sujeer M K, et al. Life-support system benefits from noise. Nature 1998; 393:127-128.
61. Kauffman S A, Johnsen S. Coevolution to the edge of chaos: coupled fitness landscapes, poised states, and coevolutionary avalanches. Journal of Theoretical Biology 1991; 149:467-505.
62. Brochard L. Breathing: does regular mean normal?[comment]. Critical Care Medicine 1998; 26:1773-1774.
63. Caminal P, Domingo L, Giraldo B F, et al. Variability analysis of the respiratory volume based on non-linear prediction methods. Medical & Biological Engineering & Computing 2004; 42:86-91.
64. Sammon M P, Bruce E N. Vagal afferent activity increases dynamical dimension of respiration in rats. Journal of Applied Physiology 1991; 70:1748-1762.
65. Khamiees M, Amoateng-Adjepong Y, Manthous C A, et al. Propofol infusion is associated with a higher rapid shallow breathing index in patients preparing to wean from mechanical ventilation. Respiratory Care 2002; 47:150-153.

1. A method of analyzing a subject's respiration comprising, performing a nonlinear analysis of a respiratory mark interval data series.

2. The method of claim 1, wherein the method is a computer implemented method.

3. The method of claim 2, further comprising the step of outputting results from the nonlinear analysis.

4. The method of claim 3, further comprising producing the respiratory data series before analyzing the series.

5. The method of claim 4, where the respiratory data series is produced from measuring the interval between each successive respiratory mark of a respiratory record.

6. A method of analyzing a subject's respiration comprising; receiving a respiratory record, wherein the record contains at least two successive respiration marks; measuring the time interval between at least two successive respiration marks producing a respiratory mark interval; performing step b) for n successive marks producing a respiratory mark interval data series; performing a nonlinear analysis on the respiratory mark interval data series; and outputting results from the nonlinear analysis.

7. The method of claim 6 wherein the method is a computer implemented method.

8. The method of claim 6, wherein receiving the respiratory record comprises receiving the respiratory record from a storage medium.

9. The method of claim 6, wherein receiving the respiratory record comprises receiving the record from a computer system.

10. The method of claim 6, wherein receiving the respiratory record comprises receiving the record from a breathing assistance system.

11. The method of claim 6, wherein receiving the respiratory record comprises receiving the respiratory record via a computer network.

12. The method of claim 6, wherein the respiration mark occurs at the start of an inspiration phase.

13. The method of claim 6, wherein the respiration mark occurs within 1 second of the start of an inspiration phase.

14. The method of claim 6, wherein the respiration mark occurs within the first observable data point of the start of an inspiration phase as identified on a respiratory trace.

15. The method of claim 6, wherein respiration mark occurs when a breathing assistance system begins assisting a breath.

16. The method of claim 6, wherein the time interval is obtained by converting a data series.

17. The method of claim 6, wherein the respiratory data series comprises at least 2, 10, 100, 1000, 10,000 or any Ni>10^{PD2i} members in the series.

18. The method of claim 6, wherein the nonlinear analysis involves using the PD2i algorithm and its Min PD2i value.

19. The method of claim 6, wherein the nonlinear analysis is the PD2i algorithm and its Mean PD2i value.

20. The method of claim 6, further comprising identifying a Mean PD2i or Min PD2i for the data series.

21. The method of claim 6, further comprising the step of comparing the Mean PD2i or Min PD2i for the data series to a ventilator removal standard.

22. The method of claim 21, wherein the ventilator removal standard is Mean or Min PD2i value less than 5, 4.5, 4.0, 3.5, 3.0, 2.5, 2.0, 1.5, 1.0, or 0.5.

23. The method of claim 21, wherein the ventilator removal standard has a Mean- or Min-PD2i value of 3.50-3.30, 3.30-3.15, 3.15-3.00, 3.00-2.85, 2.85-2.75, 2.75-2.65, 2.65-2.55, 2.55-2.45 or 2.45-2.35.

24. The method of claim 21, wherein the ventilator removal standard is determined empirically.

25. The method of claim 6 further comprising the step of recommending the removal of the subject from the ventilator if the PD2i is greater than the ventilator removal standard.

26. A method of analyzing respiration of a subject comprising, recommending to the subject the performance of a first method, the first method comprising performing a nonlinear analysis of a respiratory mark interval data series.

27. A method comprising the steps of receiving an output from a first method, the first method comprising performing a nonlinear analysis of a respiratory mark interval data series and recommending the removal of the subject from a ventilator.

28. One or more computer readable media storing program code that, upon execution by one or more computer systems, causes the computer systems to perform a first method, the first method comprising performing a nonlinear analysis of a respiratory mark interval data series.

29. The method of claim 6, further comprising performing a PD2i analysis on an RRi data series produced from an ECG from the subject.

30. A computer program product comprising a computer usable memory adapted to be executed to implement a first method, the first method comprising performing a nonlinear analysis of a respiratory mark interval data series.

31. The computer program of claim 6, comprising a logic processing module, a configuration file processing module, a data organization module, and data display organization module, that are embodied upon a computer readable medium.

32. A computer program product, comprising a computer usable medium having a computer readable program code embodied therein, said computer readable program code adapted to be executed to implement a method for generating the nonlinear analysis of a respiratory mark interval data series, said method comprising further comprising: providing a system, wherein the system comprises distinct software modules, and wherein the distinct software modules comprise a logic processing module, a configuration file processing module, a data organization module, and a data display organization module.

33. The method claim 6, further comprising a computerized system configured for performing the method.

34. The method of claim 6, further comprising the outputting of the results from the nonlinear analysis.

35. A computer-readable medium having stored thereon instructions that, when executed on a programmed processor perform a first method, the first method comprising performing a nonlinear analysis of a respiratory mark interval data series.

36. A respiratory analysis system, the system comprising: a data store capable of storing respiratory data; a system processor comprising one or more processing elements, the one or more processing elements programmed or adapted to: receive respiratory data comprising at least two successive respiration marks; store the respiratory data in the data store; measure the time interval between at least two successive respiration marks producing a respiratory mark interval; repeat step 3) for n successive marks producing a respiratory mark interval data series; identify a Mean PD2i for the data series; compare the Mean PD2i for the data series to a ventilator removal standard; and output a ventilator recommendation based upon the comparison of the Mean PD2i with the ventilator removal standard.

37. The system of claim 31, wherein the system receives the respiratory data from a breathing assistance system.

38. The system of claim 31, wherein the system receives the respiratory data via a computer network.

39. The system of claim 31, further comprising a breathing assistance system.

40. The method of claim 1, wherein the nonlinear analysis involves using the PD2i algorithm and its Min PD2i value.

41. The method of claim 1, wherein the nonlinear analysis is the PD2i algorithm and its Mean PD2i value.

42. The method of claim 1, further comprising identifying a Mean PD2i or Min PD2i for the data series.

43. The method of claim 1, further comprising the step of comparing the Mean PD2i or Min PD2i for the data series to a ventilator removal standard.

44. The method of claim 43, wherein the ventilator removal standard is Mean or Min PD2i value less than 5, 4.5, 4.0, 3.5, 3.0, 2.5, 2.0, 1.5, 1.0, or 0.5.

45. The method of claim 43, wherein the ventilator removal standard has a Mean- or Min-PD2i value of 3.50-3.30, 3.30-3.15, 3.15-3.00, 3.00-2.85, 2.85-2.75, 2.75-2.65, 2.65-2.55, 2.55-2.45 or 2.45-2.35.

46. The method of claim 43, wherein the ventilator removal standard is determined empirically.

47. The method of claim 1, further comprising performing a PD2i analysis on an RRi data series produced from an ECG from the subject.

48. The computer program of claim 1, comprising a logic processing module, a configuration file processing module, a data organization module, and data display organization module, that are embodied upon a computer readable medium.

49. The method of claim 1, further comprising a computerized system configured for performing the method.

50. The method of claim 1, further comprising the outputting of the results from the nonlinear analysis.

51. The system of claim 48, wherein the system receives the respiratory data from a breathing assistance system.

52. The system of claim 48, wherein the system receives the respiratory data via a computer network.

53. The system of claim 48, further comprising a breathing assistance system.
